(19) United States
(12) Patent Application Publication
(10) Pub. No.: US 2010/0120738 A1
(43) Pub. Date: May 13, 2010

(54) ANTI-APOPTOTIC BENZODIAZEPINE RECEPTOR LIGAND INHIBITORS

(76) Inventors: Bernard Malfroy-Camine, Arlington, MA (US); Susan Doctrow, Rosalindale, MA (US)

Correspondence Address: BOZICEVIC, FIELD & FRANCIS LLP, 1900 UNIVERSITY AVENUE, SUITE 200, EAST PALO ALTO, CA, 94303, US

(21) Appl. No.: 12/440,719

(22) PCT Filed: Sep. 11, 2007
(86) PCT No.: PCT/US07/19896

§ 371 Date: Jan. 21, 2010

Related U.S. Application Data
(60) Provisional application No. 60/844,039, filed on Sep. 11, 2006.

(51) Int. Cl. A61K 31/555; C07D 513/00; A61P 25/28; A61P 25/16

(52) U.S. Cl. 514/184; 540/145

(57) ABSTRACT

The present invention provides low molecular weight porphyrin compositions for inhibiting, preventing or delaying the binding of a ligand of a mitochondrial benzodiazepine receptor. The invention also provides pharmaceutical compositions comprising these porphyrin compositions and their use in the treatment of conditions involving the mitochondrial benzodiazepine receptor or interactions between the receptor and the mitochondrial permeability transition pore e.g., drug overdose or apoptosis including neural degeneration and radiation-induced apoptosis.

### CROSS-REFERENCE TO RELATED APPLICATIONS

This application claims priority from U.S. Ser. No. 60/844,039 filed on Sep. 11, 2006 the contents of which are incorporate herein in their entirety.

### FIELD OF THE INVENTION

The present invention relates to compositions of matter for the treatment of conditions involving the mitochondrial benzodiazepine receptor and preferably, involving interactions between the mitochondrial permeability transition pore and benzodiazepine receptor e.g., apoptosis, especially neural degeneration and radiation-induced apoptosis.

### BACKGROUND OF THE INVENTION

### Low Molecular Weight Metalloporphyrins Compounds

The synthesis and structure of a large number of orally-bioavailable low molecular weight metalloporphyrins compounds is described in International Patent Publication No. WO 2005/000854 (Jan. 6, 2005). The compounds described in this publication are effective as superoxide dismutase (SOD) and/or catalase (CAT) and/or peroxidase (POD) mimetic compounds having free radical scavenging activities and the ability to function as antioxidants.

### Apoptosis and the Mitochondrial Permeability Transition Pore

One critical step of the apoptotic process is the opening of the mitochondrial permeability transition pore (mPTP) leading to the disruption of mitochondrial membrane integrity and to the dissipation of the inner transmembrane proton gradient. Opening of the mPTP leads to mitochondrial membrane depolarization, and calcium and cytochrome C release, which ultimately leads to cell death through apoptosis. As shown in FIG. 1, the apoptotic pathway is distinct from the necrotic pathway involving reactive oxygen species (ROS) such as free radicals.

The mPTP is a polyprotein structure which is inhibited by the apoptosis-inhibitory oncoprotein Bcl-2 and which is closely associated with the mitochondrial benzodiazepine receptor (mBzR). The compound PK11195, a prototypic ligand of the 18-kDa mBzR, facilitates disruption of the inner transmembrane proton gradient of mitochondria, and apoptosis by a number of different agents, including agonists of the glucocorticoid receptor, chemotherapeutic agents (etoposide, doxorubicin), gamma irradiation, and the proapoptotic second messenger ceramide. Whereas PK11195 itself may have no cytotoxic effect, it enhances apoptosis induction by these agents. This effect is not observed for benzodiazepine diazepam, whose binding site in the mBzR differs from PK11195. PK11195 may also partially reverse Bcl-2 mediated inhibition of apoptosis via a direct effect on mitochondria.

### Neurodegenerative Diseases

Diseases involving neural cell group degeneration, such as, for example, amyotrophic lateral sclerosis (ALS), spinal muscular atrophy (SMA), Huntington's disease (HD), Parkinson's disease (PD), Alzheimer's disease (AD), dementia caused by cerebral vascular disorders, and dementia accompanied by other neuronal degeneration, are generally referred to as neurodegenerative diseases. Fundamental methods of treatment have not been established for most neurodegenerative diseases, and thus treatment methods are being sought.

The c-Jun N-Terminal Kinase (JNK or SAPK) appears to be involved in neuronal apoptosis in neurodegenerative diseases. Apoptotic neurons have enhanced phosphorylation of the transcription factor c-Jun by JNK. Additionally, neuronal c-Jun levels are elevated in response to trophic factor withdrawal, and dominant-negative forms of this transcription factor are at least partially-protective against neuronal cell death evoked by selective activation of JNKs (Eilers et al., J. Neurosci. 18, 1713-1724, 1998; Ham et al., Neuron 14, 927-939).

One approach to treating neurodegenerative diseases is considered to be the administration of factors that suppress neural cell degeneration. Administration of factors that suppress neurodegeneration is expected to be effective in treating and preventing these diseases. However, as yet virtually no such factors have been found to be actually applicable as effective therapeutic drugs.

As the factors that suppress neural cell degeneration, for example, certain dopamine receptor agonists are known to possibly have such a suppression functions. However the causal relationship between dopamine antagonists and the suppression of neural cell degeneration is unclear. Moreover, not all dopamine receptor agonists have this effect. In addition, to obtain substances effective as therapeutic drugs, the discovery of other classes of substances that can be used as anti-neurodegenerative drugs is also being sought.

### SUMMARY OF THE INVENTION

In work leading up to the present invention, the inventors sought to develop a class of biologically stable non-toxic and orally bioavailable compounds for the treatment of diseases associate with apoptosis (e.g., neurodegenerative diseases) and, more particularly, having anti-apoptotic effects.

The inventors produced and screened a series of compounds for their ability to inhibit binding of PK11195 to the mitochondrial benzodiazepine receptor and selected a series of compounds having Ki values in the micromolar and nanomolar range, preferably with a Ki value of less than about 10 μM and more preferably with a Ki value of less than about 2.5 μM, and still more preferably less than about 1.0 μM.

Using model systems for Parkinson's Disease, the inventors further demonstrated that the selected compounds have the ability to reduce staurosporine-induced PC-12 apoptosis in vitro and cytotoxicity induced by 1-methyl-4-phenylpyridinium (MPP+), the active metabolite of the Parkinsonism inducing compound MPTP, which is responsible for the destruction of the nigrostriatal pathway in primates and rodents.

The inventors also tested the efficacy of this class of compounds for protecting cells against the apoptotic effects of ionizing radiation, and demonstrated that at concentrations insufficient to induce significant necrosis of cells e.g., at low micromolar or nanomolar concentration, the compounds conferred more than about 70% protection, and preferably more than about 80% protection against the effects of ionizing radiation as determined by 4,6-diamidino-2-phenylindole (DAPI) staining of cells.

Accordingly, the present invention provides a composition for inhibiting, delaying or preventing apoptosis, said composition comprising a low molecular weight porphyrin derivative that inhibits, prevents or delays binding of a ligand of a mitochondrial benzodiazepine receptor, wherein said low molecular weight porphyrin derivative has a structure represented by Structural Formula I:

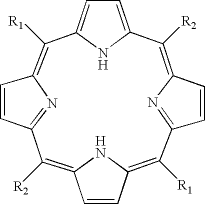

wherein one or both occurrences of R1 is aliphatic or aromatic and wherein one or both occurrences of R2 is hydrogen or aliphatic. This clearly extends to mixtures of such substitutents.

By “aliphatic” is meant a straight-chained, branched or cyclic (non-aromatic) saturated hydrocarbon. Typical straight-chained aliphatic or branched aliphatic groups have from one to about twenty carbon atoms, preferably from one to about ten carbon atoms. Typical cyclic aliphatic groups have from three to about eight ring carbon atoms. Exemplary aliphatic groups include a straight, branched chain or cyclic alkyl group e.g., methyl, ethyl, propyl, n-propyl, iso-propyl, cyclopropyl, n-butyl, iso-butyl, sec-butyl, pentyl, hexyl, cyclohexyl, octyl, cyclooctyl, methyloxy, ethyloxy, propyloxy, tetrahydropyrano, etc. The term “alkyl” refers to a hydrocarbon, including both straight-chained, cycloalkyl, groups.

By “aromatic” is meant benzyl or phenyl or a derivative thereof e.g., benzyloxy, phenoxy, methoxyphenyl, etc. or other aryl (i.e., unsubstituted or substituted aromatic hydrocarbon) substituent, the only requirement being the presence of at least one aromatic ring structure or benzene ring.

For example, R1 and/or R2 can be selected independently from the group consisting of hydrogen, methyl, ethyl, n-propyl, iso-propyl, cyclopropyl, 4-tetrahydropyrano, cyclohexyl, phenyl and 3,4-methoxyphenyl.

In another example, R1 is selected from the group consisting of aryl and lower alkyl and mixtures thereof. Alternatively, or in addition, R2 is selected from the group consisting of hydrogen, lower alkyl and mixtures thereof.

As used herein, the term “lower alkyl” shall be taken to mean an alkyl group i.e., straight-chained or cycloalkyl group, having less than about 10-12 carbon atoms. Lower alkyl groups can also have less than about 6-8 carbon atoms, or less than about 5-7 carbon atoms, or less than about 4-6 carbon atoms, or between one and about seven carbon atoms, including one or two or three or four or five or six or seven carbon atoms.

In a further example, R1 is selected from the group consisting of aryl, lower alkyl and mixtures thereof and R2 is selected from the group consisting of hydrogen, lower alkyl and mixtures thereof. In a further example, R1 is selected from aryl, lower n-alkyl, lower branched alkyl, lower cycloalkyl and mixtures thereof, and R2 is selected from hydrogen, lower n-alkyl, lower branched alkyl and mixtures thereof. Preferably, R1 consists of between one and about seven carbon atoms and R2 consists of between one and about three carbon atoms.

In a further example, R1 is benzyl, methoxyphenyl, or lower alkyl consisting of one, two, three, five or six carbon atoms or a mixture thereof and R2 is hydrogen, methyl, ethyl or a mixture thereof.

In one embodiment, the low molecular weight porphyrin derivative is complexed with a first row transition metal. Exemplary transition metals are selected from the group consisting of manganese, chromium, iron, cobalt, copper, titanium, vanadium, rubidium, osmium, nickel and zinc. In a further example, the transition metal is manganese or vanadium.

The present invention clearly includes examples wherein the porphyrin compound is complexed with an axial ligand consisting of a monovalent anion, such as, but not limited to a halogen (e.g., Cl, Br, F, I) or an organic anion (e.g., acetate, propionate, butyrate, formate, triflate). In one example, the monovalent anion is chloride or acetate.

In certain embodiments of the present invention, the low molecular weight porphyrin derivative is in a complex with a first row transition metal and a counter monovalent anion. In accordance with such examples, the low molecular weight porphyrin derivative has a structure represented by Structural Formula II (see FIG. 4 and below):

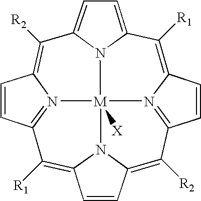

wherein:
a) each R1 is the same and selected from the group consisting of methyl, ethyl, n-propyl, iso-propyl, cyclopropyl, 4-tetrahydropyrano, cyclohexyl, phenyl and 3,4-methoxyphenyl;
b) each R2 is the same and selected from hydrogen, methyl, ethyl and iso-propyl;
c) M is a transition metal selected from the group consisting of manganese, chromium, iron, cobalt, copper, titanium, vanadium, rubidium, osmium, nickel and zinc; and
d) X is an axial ligand consisting of halogen or organic anion.

In a particularly preferred embodiment, M is manganese and X is chloride or acetate.

In a further particularly preferred embodiment, the transition metal is manganese and the axial ligand is acetate. In accordance with this example, the low molecular weight porphyrin derivative has a structure represented by Structural Formula III:

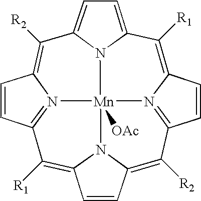

wherein:
a) each R1 is the same and selected from the group consisting of methyl, ethyl, iso-propyl, cyclopropyl, cyclohexyl, phenyl and 3,4-methoxyphenyl; and
b) each R2 is the same and selected from hydrogen, methyl, ethyl and iso-propyl.

In a further particularly preferred embodiment, the transition metal is manganese and the axial ligand is chloride. In accordance with this example, the low molecular weight porphyrin derivative has a structure represented by Structural Formula IV:

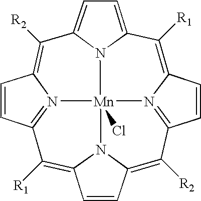

wherein:
a) each R1 is the same and selected from the group consisting of n-propyl, 4-tetrahydropyrano and cyclohexyl; and
b) each R2 is hydrogen.

Specific exemplary compounds within the present invention are selected from the group consisting of:
a) {[{(Porphine-5,15-diyl)bis[cyclopropyl-diyl]}](2-)-N²¹,N²²,N²³,N²⁴} manganese(III) acetate (EUK-418);
b) {[{(Porphine-5,15-diyl)bis[benzyl-diyl]}](2-)-N²¹,N²²,N²³,N²⁴} manganese(III) acetate (EUK-423);
c) (5,10,15,20-Tetraisopropylporphyrinato)manganese (III) acetate (EUK-424);
d) (5,10,15,20-Tetraethylporphyrinato)manganese (III) acetate (EUK-425);
e) (5,10,15,20-Tetramethylporphyrinato)manganese (III) acetate (EUK-426);
f) {[{(Porphine-5,15-diyl)bis[benzene-1,4 diyl (4-methyl-oxy)]}](2-)-N²¹,N²²,N²³,N²⁴} manganese(III) acetate (EUK-450);
g) {[{(Porphine-5,15-diyl)bis[4-Tetrahydropyrano-diyl]}](2-)-N²¹,N²²,N²³,N²⁴} manganese(III) chloride (EUK-451);
h) {[{(Porphine-5,15-diyl)bis[cyclohexyl-diyl]}](2-)-N²¹,N²²,N²³,N²⁴} manganese(III) chloride (EUK-452); and
i) {[{(Porphine-5,15-diyl)bis[propyl-diyl]}](2-)-N²¹,N²²,N²³,N²⁴} manganese(III) chloride (EUK-453).

Standard methods e.g., competition assays, are used to determine ge an inhibition, prevention or delay in binding of a ligand of a mitochondrial benzodiazepine receptor by a low molecular weight porphyrin derivative compound of the invention. As exemplified herein, labelled PK11195 compound is contacted with the receptor in the presence of different concentrations of a compound being tested and binding of the labelled PK11195 compound is determined. A reduction in binding of the labelled PK11195 to the receptor in the presence of the compound being tested indicates that the compound being tested inhibits ligands generally in their binding to the receptor. Preferably, the concentration of the compound being tested that inhibits binding of the ligand by 50% (i.e., Ki value) is determined.

In another example, the compound has moderate inhibitory activity in inhibiting ligand binding to a mitochondrial benzodiazepine receptor. By “moderate affinity” is meant that the compound inhibits the binding of the mitochondrial benzodiazepine receptor antagonist PK11195 to a mitochondrial benzodiazepine receptor at an inhibition constant (Ki) value in the low micromolar, nanomolar, picomolar or femtomolar range, e.g., less than about 5 μM, and preferably less than about 2.5 μM. Preferred compounds of the invention that inhibit ligand binding at moderate affinity to a mitochondrial benzodiazepine receptor are selected from the group consisting of:
a) {[{(Porphine-5,15-diyl)bis[cyclopropyl-diyl]}](2-)-N²¹,N²²,N²³,N²⁴} manganese(III) acetate (EUK-418);
b) {[{(Porphine-5,15-diyl)bis[benzyl-diyl]}](2-)-N²¹,N²²,N²³,N²⁴} manganese(III) acetate (EUK-423);
c) (5,10,15,20-Tetraisopropylporphyrinato)manganese (III) acetate (EUK-424); and
d) (5,10,15,20-Tetraethylporphyrinato)manganese (III) acetate (EUK-425).

Preferably, the compound inhibits ligand binding to a mitochondrial benzodiazepine receptor at high affinity. By “high affinity” is meant that the compound inhibits the binding of the mitochondrial benzodiazepine receptor antagonist PK11195 to a mitochondrial benzodiazepine receptor at an inhibition constant (Ki) value in the nanomolar, picomolar or femtomolar range e.g., less than about 1.0 μM concentration, and preferably less than about 100 nM concentration. Preferred compounds of the invention that bind at high affinity to a mitochondrial benzodiazepine receptor are selected from the group consisting of:
a) {[{(Porphine-5,15-diyl)bis[cyclopropyl-diyl]}](2-)-N²¹,N²²,N²³,N²⁴} manganese(III) acetate (EUK-418);
b) {[{(Porphine-5,15-diyl)bis[benzyl-diyl]}](2-)-N²¹,N²²,N²³,N²⁴} manganese(III) acetate (EUK-423); and
c) (5,10,15,20-Tetraethylporphyrinato)manganese (III) acetate (EUK-425).

Particularly preferred anti-apoptotic compounds of the present invention provide a protective effect again radiation-induced apoptosis and/or against STS-induced apoptosis at concentrations insufficient to induce significant necrosis i.e., toxicity, and are selected from the group consisting of:
a) {[{(Porphine-5,15-diyl)bis[cyclopropyl-diyl]}](2-)-N²¹,N²²,N²³,N²⁴} manganese(III) acetate (EUK-418);
b) {[{(Porphine-5,15-diyl)bis[benzyl-diyl]}](2-)-N²¹,N²²,N²³,N²⁴} manganese(III) acetate (EUK-423);
c) (5,10,15,20-Tetraisopropylporphyrinato)manganese (III) acetate (EUK-424);
d) (5,10,15,20-Tetraethylporphyrinato)manganese (III) acetate (EUK-425);
e) {[{(Porphine-5,15-diyl)bis[benzene-1,4 diyl (4-methyl-oxy)]}](2-)-N²¹,N²²,N²³,N²⁴} manganese(III) acetate (EUK-450);
f) {[{(Porphine-5,15-diyl)bis[4-Tetrahydropyrano-diyl]}](2-)-N²¹,N²²,N²³,N²⁴} manganese(III) chloride (EUK-451); and
g) {[{(Porphine-5,15-diyl)bis[cyclohexyl-diyl]}](2-)-N²¹,N²²,N²³,N²⁴} manganese(III) chloride (EUK-452).

In an even more preferred embodiment, an anti-apoptotic compound of the present invention provides a protective effect again radiation-induced apoptosis and/or against STS-induced apoptosis at concentrations insufficient to induce significant necrosis i.e., toxicity and is selected from the group consisting of:
a) {[{(Porphine-5,15-diyl)bis[cyclopropyl-diyl]}](2-)-N²¹,N²²,N²³,N²⁴} manganese(III) acetate (EUK-418);
b) {[{(Porphine-5,15-diyl)bis[benzyl-diyl]}](2-)-N²¹,N²²,N²³,N²⁴} manganese(III) acetate (EUK-423);
c) (5,10,15,20-Tetraethylporphyrinato)manganese (III) acetate (EUK-425);
d) {[{(Porphine-5,15-diyl)bis[benzene-1,4 diyl (4-methyl-oxy)]}](2-)-N²¹,N²²,N²³,N²⁴} manganese(III) acetate (EUK-450);
e) {[{(Porphine-5,15-diyl)bis[4-Tetrahydropyrano-diyl]}](2-)-N²¹,N²²,N²³,N²⁴} manganese(III) chloride (EUK-451); and
g) {[{(Porphine-5,15-diyl)bis[cyclohexyl-diyl]}](2-)-N²¹,N²²,N²³,N²⁴} manganese(III) chloride (EUK-452).

Preferred compounds of the present invention are non-toxic at concentrations required to produce an anti-apoptotic effect, and are preferably non-genotoxic at such concentrations by virtue of not being capable to efficiently intercalate into nucleic acid such as double-stranded DNA or to otherwise produce genotoxic side-effects. Planar molecules having aromatic substituents are more likely to be genotoxic than small non-planar molecules having aliphatic substituents. As exemplified herein, the compounds of the present invention may induce necrosis at high concentration e.g., about 10-fold to about 100-fold that required to confer protection against apoptosis. For example, about 1 μM {[{(Porphine-5,15-diyl)bis[4-Tetrahydropyrano-diyl]}](2-)-N²¹,N²²,N²³,N²⁴} manganese(III) chloride (EUK-451) provides significant protection against the apoptotic effects of ionizing radiation of isolated bovine capillary endothelial cells as determined by DAPI staining, whereas only 100 μM EUK-451 is sufficient to induce significant necrosis of such cells as determined by LDH release. Several other compounds of the present invention protect against the effects of ionizing radiation at about 1-3 μM concentration, however are only able to induce significant necrosis at 30 μM concentration or higher. Accordingly, the compound {[{(Porphine-5,15-diyl)bis[4-Tetrahydropyrano-diyl]}](2-)-N²¹,N²²,N²³,N²⁴} manganese(III) chloride (EUK-451) is particularly preferred due to its low cytotoxicity and high anti-apoptotic activity.

A preferred compound of the present invention is readily absorbed following oral administration to an animal subject e.g., a human or other animal, such as by virtue of becoming bioavailable by passing from the lumen of the alimentary canal, stomach, large intestine, small intestine or elsewhere in the digestive tract, to the bloodstream of the subject. For example, at least about 90% of a compound of the invention remains after about 90 minutes incubation in simulated gastric fluid (SGF) at a pH value of 1.2, indicating that the compounds have high resistance to acid hydrolysis and, as a consequence, the acid environment of the stomach is not a barrier to oral bioavailability. In vivo, compounds are recoverable from plasma following their oral administration to animals by intragastric gavage. These data indicate that in suitable formulations the compounds of the invention are appropriate for oral administration.

It will also be apparent from the disclosure herein that a compound of the present invention has a low molecular weight i.e., of less than about 1000 Daltons. In certain embodiments, a compound of the present invention has a molecular weight of less than about 600 Daltons, or between about 400 Daltons and about 600 Daltons. In a further example, a compound of the present invention has molecular weight of between about 400 Daltons and about 1000 Daltons.

Certain compounds disclosed herein have not been disclosed previously as specific compositions of matter, in particular methoxyphenyl, tetrahydropyrano, cyclohexyl and n-propyl metalloporphyrin derivatives.

Accordingly, another example of the present invention provides a composition comprising a low molecular weight methoxyphenyl porphyrin derivative having a structure represented by Structural Formula I:

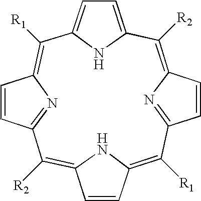

wherein R1 and/or R2 are each methoxyphenyl subject to the proviso that when R1 and R2 are not both methoxyphenyl then R1 or R2 is hydrogen.

Preferred methoxyphenyl derivatives of the invention have a structure represented by Structural Formula II:

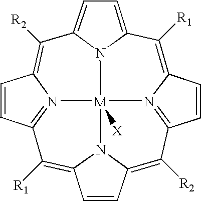

wherein:
a) R1 and/or R2 are each methoxyphenyl subject to the proviso that when R1 and R2 are not both methoxyphenyl then R1 or R2 is hydrogen;
b) M is a transition metal selected from the group consisting of manganese, chromium, iron, cobalt, copper, titanium, vanadium, rubidium, osmium, nickel and zinc; and
c) X is an axial ligand consisting of halogen or organic anion.

More preferably, M is manganese and X is chloride or acetate.

Even more preferably, M is manganese and X is acetate.

In a particularly preferred example, the methoxyphenyl porphyrin derivative is {[{(Porphine-5,15-diyl)bis[benzene-1,4 diyl (4-methyl-oxy)]}](2-)-N²¹,N²²,N²³,N²⁴} manganese(III) acetate (EUK-450).

In yet another example, the present invention provides a composition comprising a low molecular weight tetrahydropyrano porphyrin derivative having a structure represented by Structural Formula I:

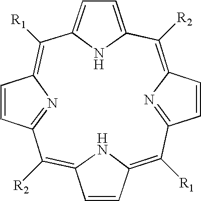

wherein R1 and/or R2 are each 4-tetrahydropyrano subject to the proviso that when R1 and R2 are not both 4-tetrahydropyrano then R1 or R2 is hydrogen.

Preferred 4-tetrahydropyrano derivatives of the invention have a structure represented by Structural Formula II:

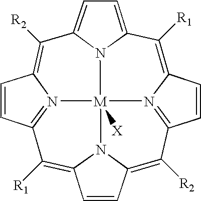

wherein:
a) R1 and/or R2 are each 4-tetrahydropyrano subject to the proviso that when R1 and R2 are not both 4-tetrahydropyrano then R1 or R2 is hydrogen;
b) M is a transition metal selected from the group consisting of manganese, chromium, iron, cobalt, copper, titanium, vanadium, rubidium, osmium, nickel and zinc; and
c) X is an axial ligand consisting of halogen or organic anion.

More preferably, M is manganese and X is chloride or acetate.

Even more preferably, M is manganese and X is chloride.

In a particularly preferred example of the invention, the 4-tetrahydropyrano derivative is {[{(Porphine-5,15-diyl)bis[4-Tetrahydropyrano-diyl]}](2-)-N²¹,N²²,N²³,N²⁴} manganese(III) chloride (EUK-451).

In yet another example, the present invention provides a composition comprising a low molecular weight cyclohexyl porphyrin derivative having a structure represented by Structural Formula I:

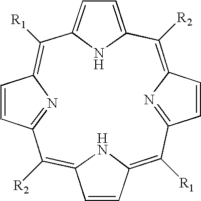

wherein R1 and/or R2 are each cyclohexyl subject to the proviso that when R1 and R2 are not both cyclohexyl then R1 or R2 is hydrogen.

Preferred cyclohexyl derivatives of the invention have a structure represented by Structural Formula II:

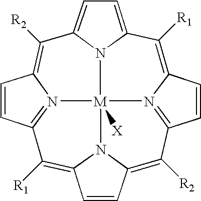

wherein:
a) R1 and/or R2 are each cyclohexyl subject to the proviso that when R1 and R2 are not both cyclohexyl then R1 or R2 is hydrogen;
b) M is a transition metal selected from the group consisting of manganese, chromium, iron, cobalt, copper, titanium, vanadium, rubidium, osmium, nickel and zinc; and
c) X is an axial ligand consisting of halogen or organic anion.

More preferably, M is manganese and X is chloride or acetate.

Even more preferably, M is manganese and X is chloride.

In a particularly preferred example of the invention, the cyclohexyl derivative is {[{(Porphine-5,15-diyl)bis[cyclohexyl-diyl]}](2-)-N²¹,N²²,N²³,N²⁴} manganese (III) chloride (EUK-452).

In yet another example the present invention provides a composition comprising a low molecular weight n-propyl porphyrin derivative having a structure represented by Structural Formula I:

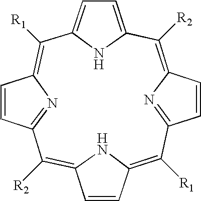

wherein R1 and/or R2 are each n-propyl subject to the proviso that when R1 and R2 are not both n-propyl then R1 or R2 is hydrogen.

Preferred n-propyl derivatives of the invention have a structure represented by Structural Formula II:

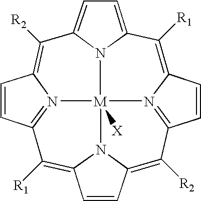

wherein:
a) R1 and/or R2 are each n-propyl subject to the proviso that when R1 and R2 are not both n-propyl then R1 or R2 is hydrogen;
b) M is a transition metal selected from the group consisting of manganese, chromium, iron, cobalt, copper, titanium, vanadium, rubidium, osmium, nickel and zinc; and
c) X is an axial ligand consisting of halogen or organic anion.

More preferably, M is manganese and X is chloride or acetate.

Even more preferably, M is manganese and X is chloride.

In a particularly preferred embodiment, the n-propyl porphyrin derivative of the present invention is {[{(Porphine-5,15-diyl)bis[propyl-diyl]}](2-)-N²¹,N²²,N²³,N²⁴} manganese(III) chloride (EUK-453).

Analogs of the compounds exemplified herein are encompassed by the invention. Preferred analogs of the compounds of the present invention have substitutions at the R1 and R2 positions i.e., C-5, C-10, C-15 and C-20. It is preferred that such analogs have sufficient stability, solubility and oral bioavailability, and sufficiently low toxicity, to permit their use as pharmaceutical agents in the treatment of free-radical associated disease and/or apoptosis-associated disease. Preferred analogs will possess lower toxicity and/or enhanced oral bioavailability and/or enhanced solubility compared to the compounds from which they are derived. It is also preferred that such analogs inhibit, delay or prevent ligand binding to a peripheral BZD receptor e.g., as determined using the exemplified PK11195 binding assay described herein.

In one example, a preferred compound according to any embodiment supra will inhibit, prevent or reduce opening of a mitochondrial permeability transition pore (mPTP) in a cell.

In another example, a preferred compound according to any embodiment supra will inhibit, prevent or reduce mitochondrial membrane depolarization in a cell.

In another example, a preferred compound according to any embodiment supra will inhibit, prevent or reduce the release of calcium and/or cytochrome C from a cell.

The present invention also provides a pharmaceutical formulation comprising one or more pharmaceutically acceptable carriers, diluents or excipients and a therapeutically effective amount of at least one low molecular weight porphyrin derivative compound described herein with respect to any embodiment supra. including a compound having the structure of Formula I or II or III or IV or as exemplified in any one or more of FIGS. 2-10. Preferred pharmaceutical compositions of the invention will comprise the low molecular weight porphyrin derivative compound in a therapeutically effective amount to prevent, delay or inhibit apoptosis and/or inhibit, prevent or reduce opening of a mitochondrial permeability transition pore (mPTP) in a cell and/or inhibit, prevent or reduce mitochondrial membrane depolarization in a cell and/or inhibit, prevent or reduce the release of calcium and/or cytochrome C from a cell.

The present invention also provides a method of treating a disease associated with apoptosis in a mammal said method comprising administering to the mammal an amount of a pharmaceutical formulation of the present invention effective to inhibit, delay or prevent apoptosis.

The present invention also provides for a use of a composition of the present invention in the preparation of a medicament for the treatment of a disease associated with apoptosis in a mammal.

As will be apparent to the skilled artisan, the compounds and pharmaceutical compositions of the invention, possess such utility by virtue of their ability to inhibit the binding of a ligand of the benzodiazepine receptor and/or inhibit, prevent or reduce opening of a mitochondrial permeability transition pore (mPTP) in a cell and/or inhibit, prevent or reduce mitochondrial membrane depolarization in a cell and/or inhibit, prevent or reduce the release of calcium and/or cytochrome C from a cell. Without being bound by any theory or mode of action, the efficacy of the compounds of the present invention also resides in their ability to block, inhibit or reduce opening of the mPTP or otherwise prevent the efflux of calcium and/or cytochrome C that would lead to apoptosis. Thus, the present invention is not limited in scope by the nature of any disease to be treated other than a requirement for aetiology and/or progression of the disease to be associated with apoptosis, and/or for the severity of one or more disease symptoms to be associated with apoptosis. Diseases for which the present invention is particularly useful in treating include neurodegenerative diseases e.g., diseases selected from the group consisting of Alzheimer's disease, dementia, Parkinson's disease, Lou Gehrig disease, motor neuron disease, Huntington's disease and multiple sclerosis. The treatment of Parkinson's Disease is preferred.

Additionally, as exemplified herein the compounds and pharmaceutical compositions of the present invention are useful for treating radiation-induced apoptosis. Accordingly, the present invention also provides a method of treating radiation-induced apoptosis in a mammal said method comprising administering to the mammal an amount of a pharmaceutical formulation of the present invention effective to inhibit, delay or prevent radiation-induced apoptosis.

The present invention also provides for a use of a composition of the present invention in the preparation of a medicament for the treatment of radiation-induced apoptosis in a mammal.

Additionally, the compounds and pharmaceutical compositions of the present invention of the present invention are useful for treating the adverse effects of a mitochondrial benzodiazepine receptor ligand i.e., agonist or antagonist, by virtue of their being able to compete binding of the ligand to the receptor. Such applications relate to the treatment of drug overdose. Accordingly, the present invention also provides a method of treating an adverse effect of a mitochondrial benzodiazepine receptor ligand said method comprising administering to the mammal an amount of a pharmaceutical formulation of the present invention effective to inhibit, delay or prevent binding of the ligand to the receptor. The ligand may be an agonist of the receptor or an antagonist of the receptor.

The present invention also provides for a use of a composition of the present invention in the preparation of a medicament for the treatment of an adverse effect of a mitochondrial benzodiazepine receptor ligand.

### BRIEF DESCRIPTION OF THE DRAWINGS

FIG. 1 is a schematic representation showing the distinction between necrotic and apoptotic pathways in mitochondria.

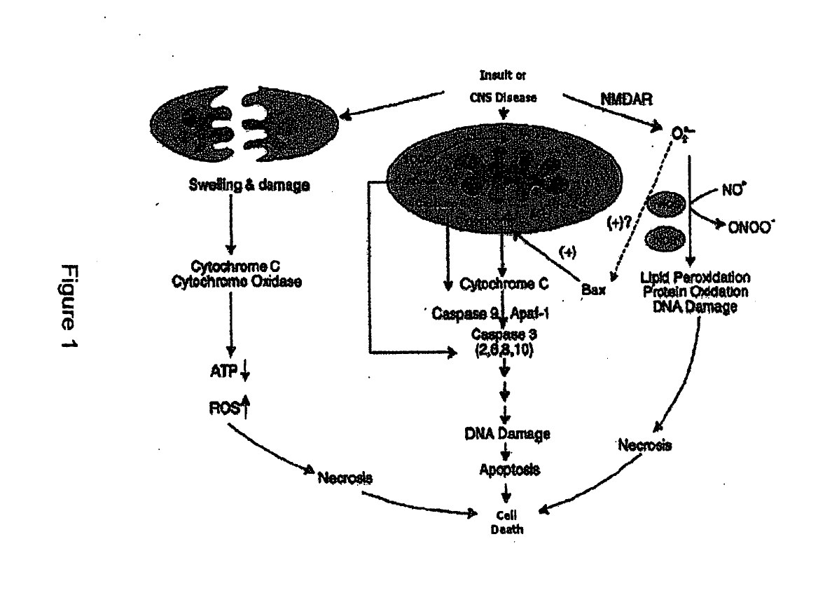

FIG. 2 is a schematic representation showing a preferred means for synthesis of {[{(Porphine-5,15-diyl)bis[cyclopropyl-diyl]}](2-)-N²¹,N²²,N²³,N²⁴} manganese(III) acetate (EUK-418).

FIG. 3 is a schematic representation showing a preferred means for synthesis of {[{(Porphine-5,15-diyl)bis[benzyl-diyl]}](2-)-N²¹,N²²,N²³,N²⁴} manganese(III) acetate (EUK-423).

FIG. 4 is a schematic representation showing a preferred means for synthesis of (5,10,15,20-Tetraisopropylporphyrinato)manganese (III) acetate (EUK-424).

FIG. 5 is a schematic representation showing a preferred means for synthesis of (5,10,15,20-Tetraethylporphyrinato)manganese (III) acetate (EUK-425).

FIG. 6 is a schematic representation showing a preferred means for synthesis of (5,10,15,20-Tetramethylporphyrinato)manganese (III) acetate (EUK-426).

FIG. 7 is a schematic representation showing a preferred means for synthesis of {[{(Porphine-5,15-diyl)bis[benzene-1,4 diyl (4-methyl-oxy)]}](2-)-N²¹,N²²,N²³,N²⁴} manganese(III) acetate (EUK-450).

FIG. 8 is a schematic representation showing a preferred means for synthesis of {[{(Porphine-5,15-diyl)bis[4-Tetrahydropyrano-diyl]}](2-)-N²¹,N²²,N²³,N²⁴} manganese(III) chloride (EUK-451).

FIG. 9 is a schematic representation showing a preferred means for synthesis of {[{(Porphine-5,15-diyl)bis[cyclohexyl-diyl]}](2-)-N²¹,N²²,N²³,N²⁴} manganese(III) chloride (EUK-452).

FIG. 10 is a schematic representation showing a preferred means for synthesis of {[{(Porphine-5,15-diyl)bis[propyl-diyl]}](2-)-N²¹,N²²,N²³,N²⁴} manganese(III) chloride (EUK-453).

FIG. 11 is a graphical representation showing the effect of the low molecular weight metalloporphyrins compounds {[{(Porphine-5,15-diyl)bis[cyclopropyl-diyl]}](2-)-N²¹,N²²,N²³,N²⁴} manganese(III) acetate (EUK-418) and {[{(Porphine-5,15-diyl)bis[benzyl-diyl]}](2-)-N²¹,N²²,N²³,N²⁴} manganese(III) acetate (EUK-423) on staurosporine-induced apoptosis of PC12 cells in vitro. Data indicate that both compounds effectively reduce apoptosis.

FIG. 12 is a graphical representation showing the effect of the low molecular weight metalloporphyrins compounds {[{(Porphine-5,15-diyl)bis[cyclopropyl-diyl]}](2-)-N²¹,N²²,N²³,N²⁴} manganese(III) acetate (EUK-418), {[{(Porphine-5,15-diyl)bis[benzyl-diyl]}] (2-)-N²¹,N²²,N²³,N²⁴} manganese(III) acetate (EUK-423), (5,10,15,20-Tetra ethylporphyrinato)manganese (III) acetate (EUK-425), {[{(Porphine-5,15-diyl)bis[benzene-1,4 diyl (4-methyl-oxy)]}] (2-)-N²¹,N²²,N²³,N²⁴} manganese(III) acetate (EUK-450), {[{(Porphine-5,15-diyl)bis[4-Tetrahydropyrano-diyl]}](2-)-N²¹,N²²,N²³,N²⁴} manganese(III) chloride (EUK-451), {[{(Porphine-5,15-diyl)bis[cyclohexyl-diyl]}](2-)-N²¹,N²²,N²³,N²⁴} manganese(III) chloride (EUK-452), and {[{(Porphine-5,15-diyl)bis[propyl-diyl]}](2-)-N²¹,N²²,N²³,N²⁴} manganese(III) chloride (EUK-453) on staurosporine-induced apoptosis of PC12 cells in vitro. Data indicate that the compounds effectively reduce apoptosis at up to about 5 μM concentration, however in this cellular model, toxicity blunts protection at high concentrations of the compounds.

FIG. 13 is a graphical representation showing mitigation of radiation-induced apoptosis in bovine capillary endothelial cells conferred by {[{(Porphine-5,15-diyl)bis[cyclopropyl-diyl]}](2-)-N²¹,N²²,N²³,N²⁴} manganese(III) acetate (EUK-418). Bovine capillary endothelial cells were cultured on eight chamber Labtek slides and exposed to ionizing radiation (20 Gy) for 6 hours. After this time, cells were either left untreated or treated with compound at the concentration indicated on the x-axis. Cells were then fixed in methanol and stained with 5 μg/ml DAPI and DNA was visualized using a Nikon epifluorescence microscope, and apoptosis scored and expressed as an apoptotic index according to the percentage of apoptotic cells in a field of 100 cells. Open bars indicate apoptotic index following irradiation. Hatched bars indicate apoptotic index for control cells not receiving a dose of ionizing radiation. Data indicate that about 3 μM of the compound EUK-418 provides significant protection from the apoptotic effects of ionizing radiation in this model. **, p<0.0001.

FIG. 14 is a graphical representation showing mitigation of radiation-induced apoptosis in bovine capillary endothelial cells conferred by {[{(Porphine-5,15-diyl)bis[benzyl-diyl]}](2-)-N²¹,N²²,N²³,N²⁴} manganese(III) acetate (EUK-423). Bovine capillary endothelial cells were cultured on eight chamber Labtek slides and exposed to ionizing radiation (20 Gy) for 6 hours. After this time, cells were either left untreated or treated with compound at the concentration indicated on the x-axis. Cells were then fixed in methanol and stained with 5 μg/ml DAPI and DNA was visualized using a Nikon epifluorescence microscope, and apoptosis scored and expressed as an apoptotic index according to the percentage of apoptotic cells in a field of 100 cells. Open bars indicate apoptotic index following irradiation. Hatched bars indicate apoptotic index for control cells not receiving a dose of ionizing radiation. Data indicate that about 3-10 μM of the compound EUK-423 provides significant protection from the apoptotic effects of ionizing radiation in this model. *, p<0.006.

FIG. 15 is a graphical representation showing the ability of the compounds {[{(Porphine-5,15-diyl)bis[cyclopropyl-diyl]}](2-)-N²¹,N²²,N²³,N²⁴} manganese (III) acetate (EUK-418) and (5,10,15,20-Tetra ethylporphyrinato) manganese (III) acetate (EUK-425) to inhibit binding of tritiated PK11195 to the mitochondrial benzodiazepine receptor (mBzR). The Ki value for each compound is indicated.

FIG. 16 is a tabular representation showing a series of low molecular weight metalloporphyrin compounds designated EUK-418, EUK-423, EUK-424, EUK-425 and EUK-426, that inhibit ligand binding to the mitochondrial benzodiazepine receptor (mBzR) with moderate affinity, as determined by the Ki value (last column) for inhibition of tritiated PK11195, binding to the receptor. The first two columns of the table show the substituents R1 and R2 For each compound indicated, the R1 substituents are the same, and the R2 substituents are the same. M represents a transition metal, which is manganese for the compounds indicated. X represents a counter monovalent anion, which is acetate (OAc) for the compounds indicated. The general structure of compounds (i.e., Formula II) is also indicated below the table.

FIG. 17 is a graphical representation showing the effects of exemplary low molecular weight metalloporphyrin compounds of the present invention on MPP+ induced neurodegeneration in cultured mesencephalic tissue slices. Mesencephalic tissue slices were prepared from PND 3-5 rats and maintained in culture for 2 weeks. The tissue slices were incubated with a porphyrin compound as indicated on the x-axis for 6 hours before adding 20 μM MPP+ to induce neuronal apoptosis. Concentrations of each porphyrin compound tested were 100 nM [symbol] 1.0 μM [symbol] and 10 μM [symbol] Following a further incubation for 48 hours, the slices were collected and the level of lactate dehydrogenase (LDH) released into the medium was determined. Data indicate the mean±SEM for 4-6 experiments.

FIG. 18 is a graphical representation showing the bioavailability in vivo for the compounds {[{(Porphine-5,15-diyl)bis[cyclopropyl-diyl]}](2-)-N²¹,N²²,N²³,N²⁴} manganese(III) acetate (EUK-418) and {[{(Porphine-5,15-diyl)bis[benzyl-diyl]}](2-)-N²¹,N²²,N²³,N²⁴} manganese(III) acetate (EUK-423) in rats. Fasted and fed Sprague-Dawley rats were dosed by intragastric gavage with either 4 mg/kg EUK-418 or 2 mg/kg EUK-423. At the times indicated on the x-axis, plasma was obtained from the animals and the concentrations of compounds determined by LC-MS/MS. Data indicate that both compounds are bioavailable in vivo. EUK-423 increased in plasma during the first four hour period following intragastric gavage with the compound, whereas EUK-418 increased rapidly in serum of fasted rats and then declined to about 100 ng/ml concentration for the assayed period. Fasting of animals also appeared to increase plasma concentration of EUK-418.

FIG. 19 is a graphical representation showing catalase activity for a 10 μM concentration of the compounds indicated on the x-axis, as determined by measuring H₂O₂ degradation per minute (y-axis). Data indicate significant catalase activity for the compounds tested. Error bars show standard deviations of triplicate samples.

### DETAILED DESCRIPTION OF THE PREFERRED EMBODIMENTS

### Formulations

While it is possible for the compounds of the present invention to be administered as the complex per se, it is preferred to present the compounds or the complexes in the form of a pharmaceutical formulation.

Formulation of a pharmaceutical compound will vary according to the route of administration selected (e.g., solution, emulsion, capsule). For solutions or emulsions, suitable carriers include, for example, aqueous or alcoholic/aqueous solutions, emulsions or suspensions, including saline and buffered media. Parenteral vehicles can include sodium chloride solution, Ringer's dextrose, dextrose and sodium chloride, lactated Ringer's or fixed oils, for instance. Intravenous vehicles can include various additives, preservatives, or fluid, nutrient or electrolyte replenishers and the like (See, generally, Remington's Pharmaceutical Sciences, 17th Edition, Mack Publishing Co., Pa., 1985). For inhalation, the agent can be solubilized and loaded into a suitable dispenser for administration (e.g., an atomizer, nebulizer or pressurized aerosol dispenser).

Pharmaceutical formulations can be adapted for administration by any appropriate route, for example by the oral (including buccal or sublingual), rectal, nasal, topical (including buccal, sublingual or transferal), vaginal or parenteral (including subcutaneous, intramuscular, intravenous or intradermal) route. Such formulations can be prepared by any method known in the art of pharmacy, for example by bringing into association the active ingredient with the carrier(s), diluent(s) or excipient(s).

To prepare such pharmaceutical formulations, one or more compounds of the present invention is/are mixed with a pharmaceutically acceptable carrier or excipient for example, by mixing with physiologically acceptable carriers, excipients, or stabilizers in the form of, e.g., lyophilized powders, slurries, aqueous solutions, or suspensions (see, e.g., Hardman, et al. (2001) Goodman and Gilman's The Pharmacological Basis of Therapeutics, McGraw-Hill, New York, N.Y.; Gennaro (2000) Remington: The Science and Practice of Pharmacy, Lippincott, Williams, and Wilkins, New York, N.Y.; Avis, et al. (eds.) (1993) Pharmaceutical Dosage Forms: Parenteral Medications, Marcel Dekker, NY; Lieberman, et al. (eds.) (1990) Pharmaceutical Dosage Forms: Tablets, Marcel Dekker, NY; Lieberman, et al. (eds.) (1990) Pharmaceutical Dosage Forms: Disperse Systems, Marcel Dekker, NY; Weiner and Kotkoskie (2000) Excipient Toxicity and Safety, Marcel Dekker, Inc., New York, N.Y.).

As will be apparent to a skilled artisan, a compound that is active in vivo is particularly preferred. A compound that is active in a human subject is even more preferred. Accordingly, when manufacturing a compound that is useful for the treatment of a disease it is preferable to ensure that any components added to the formulation do not inhibit or modify the activity of the active compound.

Pharmaceutical formulations may be presented in unit dose forms containing a predetermined amount of active ingredient per unit dose. Such a unit may contain for example 1 μg to 10 ug, such as 0.01 mg to 1000 mg, or 0.1 mg to 250 mg, of a compound of Structural Formula I, Structural Formula II, Structural Formula III or Structural Formula IV, depending on the condition being treated, the route of administration and the age, weight and condition of the patient.

### a) Oral Formulations

Pharmaceutical formulations adapted for oral administration may be presented as discrete units such as capsules, soft gels, or tablets; powders or granules; solutions or suspensions in aqueous or non-aqueous liquids; edible foams or whips; or oil-in-water liquid emulsions or water-in-oil liquid emulsions. Particularly preferred oral formulations account for the relative lipophilicity of the compounds of Structures I-IV.

In general. formulations suitable for oral steroid compositions are suitable oral formulations for the metalloporphyrin derivatives of the present invention:

Granular Tablets and Capsules

In one example, the oral formulation comprises an intragranular phase comprising an effective amount of a metalloporphyrin derivative of the present invention and at least one carbohydrate alcohol and an aqueous binder. Preferably, the pharmaceutical formulation is substantially lactose-free. Preferred carbohydrate alcohols for such formulations are selected from the group consisting of mannitol, maltitol, sorbitol, lactitol, erythritol and xylitol. Preferably, the carbohydrate alcohol is present at a concentration of about 15% to about 90%. A preferred aqueous binder is selected from the group consisting of hydroxypropyl cellulose, hydroxypropyl methylcellulose, carboxymethyl cellulose sodium, polyvinyl pyrrolidones, starches, gelatins and povidones. A binder is generally present in the range of from about 1% to about 15%. The intragranular phase can also comprise one or more diluents, such as, for example, a diluent selected from the group consisting of microcrystalline cellulose, powdered cellulose, calcium phosphate-dibasic, calcium sulfate, dextrates, dextrins, alginates and dextrose excipients. Such diluents are also present in the range of about 15% to about 90%. The intragranular phase can also comprise one or more disintegrants, such as, for example, a disintegrant selected from the group consisting of a low substituted hydroxypropyl cellulose, carboxymethyl cellulose, calcium carboxymethylcellulose, sodium carboxymethyl cellulose, sodium starch glycollate, crospovidone, croscarmellose sodium, starch, crystalline cellulose, hydroxypropyl starch, and partially pregelatinized starch. A disintegrant is generally present in the range of from about 5% to about 20%. Such a formulation can also comprise one or more lubricants such as, for example, a lubricant selected from the group consisting of talc, magnesium stearate, stearic acid, hydrogenated vegetable oils, glyceryl behenate, polyethylene glycols and derivatives thereof, sodium lauryl sulphate and sodium stearyl fumarate. A lubricant is generally present in the range of from about 0.5% to about 5%. Such formulations are made into a tablet, capsule, or soft gel e.g., by a process comprising mixing a metalloporphyrin derivative of the invention and at least one carbohydrate alcohol to form a dry blend, wet granulating the dry blend with an aqueous binder so as to obtain an intragranular phase, and further formulating the resulting intragranular phase so as to provide the formulation. Typically, tablet or capsules will be prepared to contain from 1 mg to 1000 mg, such as 2.5 mg to 250 mg of active ingredient per unit dose.

Hard or Soft Gels

A liquid or semi-solid pharmaceutical formulation for oral administration e.g., a hard gel or soft gel capsule, may be prepared comprising:

(a) a first carrier component comprising from about 10% to about 99.99% by weight of a metalloporphyrin derivative of the present invention;
(b) an optional second carrier component comprising, when present, up to about 70% by weight of said metalloporphyrin derivative;
(c) an optional emulsifying/solubilizing component comprising, when present, from about 0.01% to about 30% by weight of said metalloporphyrin derivative;
(d) an optional anti-crystallization/solubilizing component comprising, when present, from about 0.01% to about 30% by weight of said metalloporphyrin derivative; and
(e) an active pharmacological agent comprising from about 0.01% to about 80% of said metalloporphyrin derivative in anhydrous crystal form.

The first carrier component and optional second carrier component generally comprise, independently, one or more of lauroyl macrogol glycerides, caprylocaproyl macrogolglycerides, stearoyl macrogol glycerides, linoleoyl macrogol glycerides, oleoyl macrogol glycerides, polyalkylene glycol, polyethylene glycol, polypropylene glycol, polyoxyethylene-polyoxypropylene copolymer, fatty alcohol, polyoxyethylene fatty alcohol ether, fatty acid, polyethoxylated fatty acid ester, propylene glycol fatty acid ester, fatty ester, glycerides of fatty acid, polyoxyethylene-glycerol fatty ester, polyoxypropylene-glycerol fatty ester, polyglycolized glycerides, polyglycerol fatty acid ester, sorbitan ester, polyethoxylated sorbitan ester, polyethoxylated cholesterol, polyethoxylated castor oil, polyethoxylated sterol, lecithin, glycerol, sorbic acid, sorbitol, or polyethoxylated vegetable oil.

The emulsifying/solubilizing component generally comprises one or more of metallic alkyl sulfate, quaternary ammonium compounds, salts of fatty acids, sulfosuccinates, taurates, amino acids, lauroyl macrogol glycerides, caprylocaproyl macrogolglycerides, stearoyl macrogol glycerides, linoleoyl macrogol glycerides, oleoyl macrogol glycerides, polyalkylene glycol, polyethylene glycol, polypropylene glycol, polyoxyethylene-polyoxypropylene copolymer, polyoxyethylene fatty alcohol ether, fatty acid, polyethoxylated fatty acid ester, propylene glycol fatty acid ester, polyoxyethylene-glycerol fatty ester, polyglycolized glycerides, polyglycerol fatty acid ester, sorbitan ester, polyethoxylated sorbitan ester, polyethoxylated cholesterol, polyethoxylated castor oil, polyethoxylated sterol, lecithin, or polyethoxylated vegetable oil. The anti-crystallization/solubilizing component, when present, generally comprises one or more of metallic alkyl sulfate, polyvinylpyrrolidone, lauroyl macrogol glycerides, caprylocaproyl macrogolglycerides, stearoyl macrogol glycerides, linoleoyl macrogol glycerides, oleoyl macrogol glycerides, polyalkylene glycol, polyethylene glycol, polypropylene glycol, polyoxyethylene-polyoxypropylene copolymer, fatty alcohol, polyoxyethylene fatty alcohol ether, fatty acid, polyethoxylated fatty acid ester, propylene glycol fatty acid ester, fatty ester, glycerides of fatty acid, polyoxyethylene-glycerol fatty ester, polyglycolized glycerides, polyglycerol fatty acid ester, sorbitan ester, polyethoxylated sorbitan ester, polyethoxylated cholesterol, polyethoxylated castor oil, polyethoxylated sterol, lecithin, or polyethoxylated vegetable oil.

Bioadhesive Polymeric Formulations

Particularly preferred formulations for oral delivery of a metalloporphyrinderivative of the invention account for its relative lipophilicity and ready absorption by the lining of the stomach and/or the intestine. By appropriate formulation of the compounds, their levels in body fluids such as plasma and urine can be enhanced, relative to their deposition in adipose tissues.

For example, a metalloporphyrin of the invention is formulated with a hydrophobic polymer, preferably a bioadhesive polymer and optionally encapsulated in or dispersed throughout a microparticle or nanoparticle. The bioadhesive polymer improves gastrointestinal retention via adherence of the formulation to the walls of the gastrointestinal tract. Suitable bioadhesive polymers include polylactic acid, polystyrene, poly(bis carboxy phenoxy propane-co-sebacic anhydride) (20:80) (poly (CCP:SA)), alginate (freshly prepared); and poly(fumaric anhydride-co-sebacic anhydride (20:80) (poly (FA:SA)), types A (containing sudan red dye) and B (undyed). Other high-adhesion polymers include p(FA:SA) (50:50) and non-water-soluble polyacrylates and polyacrylamides. Preferred bioadhesive polymers are typically hydrophobic enough to be non-water-soluble, but contain a sufficient amount of exposed surface carboxyl groups to promote adhesion e.g., non-water-soluble polyacrylates and polymethacrylates; polymers of hydroxy acids, such as polylactide and polyglycolide; polyanhydrides; polyorthoesters; blends comprising these polymers; and copolymers comprising the monomers of these polymers. Preferred biopolymers are bioerodable, with preferred molecular weights ranging from 1000 to 15,000 kDa, and most preferably 2000 to 5000 Da. Polyanhydrides e.g., polyadipic anhydride (“p(AA)”), polyfumaric anhydride, polysebacic anhydride, polymaleic anhydride, polymalic anhydride, polyphthalic anhydride, polyisophthalic anhydride, polyaspartic anhydride, polyterephthalic anhydride, polyisophthalic anhydride, poly carboxyphenoxypropane anhydride and copolymers with other polyanhydrides at different mole ratios, are particularly preferred.

Blends of hydrophilic polymers and bioadhesive hydrophobic polymers can also be employed. Suitable hydrophilic polymers include e.g., hydroxypropylmethylcellulose, hydroxypropylcellulose, carboxymethylcellulose, polyvinylalcohols, polyvinylpyrollidones, and polyethylene glycols.

Other mucoadhesive polymers include DOPA-maleic anhydride co polymer, isopthalic anhydride polymer, DOPA-methacrylate polymers, DOPA-cellulosic based polymers, and DOPA-acrylic acid polymers.

Excipients will typically be included in the dosage form e.g., to improve bioadhesion. Suitable excipients include solvents, co-solvents, emulsifiers, plasticizers, surfactants, thickeners, pH modifiers, emollients, antioxidants, and chelating agents, wetting agents, and water absorbing agents. The formulation may also include one or more additives, for example, dyes, colored pigments, pearlescent agents, deodorizers, and odor maskers.

The metalloporphyrin may optionally be encapsulated or molecularly dispersed in polymers to reduce particle size and increase dissolution. The polymers may include polyesters such as polylactic acid) or P(LA), polycaprylactone, polylactide-coglycolide or P(LGA), poly hydroxybutyrate poly β-malic acid); polyanhydrides such as poly(adipic)anhydride or P(AA), poly(fumaric-co-sebacic)anhydride or P(FA:SA), poly(sebacic)anhydride or P(SA); cellulosic polymers such as ethylcellulose, cellulose acetate, cellulose acetate phthalate, etc; acrylate and methacrylate polymers such as Eudragit RS 100, RL 100, E100 PO, L100-55, L100, S100 (distributed by Rohm America) or other polymers commonly used for encapsulation for pharmaceutical purposes and known to those skilled in the art. Also suitable are hydrophobic polymers such as polyimides.

Blending or copolymerization sufficient to provide a certain amount of hydrophilic character can be useful to improve wettability of the materials. For example, about 5% to about 20% of monomers may be hydrophilic monomers. Hydrophilic polymers such as hydroxylpropylcellulose (HPC), hydroxypropylmethylcellulose (HPMC), carboxymethylcellulose (CMC) are commonly used for this purpose.

The formulation may be an “immediate release” formulation that releases at least 85% (wt/wt) of the active metalloporphyrin derivative within 60 minutes in vitro. Alternatively, the formulation is a “controlled release” formulation that releases drug more slowly than an immediate release formulation i.e., it takes longer than 60 minutes to release at least 85% (wt/wt) of the drug in vitro. To extend the time period for release, the ratio of metalloporphyrin derivative to polymer can be increased. Increased relative drug concentration is believed to have the effect of increasing the effective compound domain size within the polymer matrix thereby slowing dissolution. In the case of a polymer matrix containing certain types of hydrophobic polymers, the polymer will act as a mucoadhesive material and increase the retention time of the active compound in the gastrointestinal tract. Increased drug dissolution rates combined with the mucoadhesive properties of the polymer matrix increase uptake of the active compound and reduce differences found in the fed and fasted states for the compounds.

The oral formulations may be prepared using a pharmaceutically acceptable carrier composed of materials that are considered safe and effective and may be administered to an individual without causing undesirable biological side effects or unwanted interactions. Exemplary carrier include diluents, binders, lubricants, disintegrants, stabilizers, surfactants, colorants, and fillers.

Diluents or fillers increase the bulk of a solid dosage form so that a practical size is provided for compression of tablets or formation of beads and granules. Suitable diluents include, but are not limited to dicalcium phosphate dihydrate, calcium sulfate, lactose, sucrose, mannitol, sorbitol, cellulose, microcrystalline cellulose, kaolin, sodium chloride, dry starch, hydrolyzed starches, pregelatinized starch, silicone dioxide, titanium oxide, magnesium aluminum silicate and powdered sugar.

Dispersants include phosphate-buffered saline (PBS), saline, glucose, sodium lauryl sulfate (SLS), polyvinylpyrrolidone (PVP), polyethylene glycol (PEG), and hydroxypropylmethylcellulose (HPMC).

Binders may impart cohesive qualities to a solid dosage formulation, and thus ensure that a tablet, bead or granule remains intact after the formation of the dosage forms. Suitable binder materials include, but are not limited to, starch, pregelatinized starch, gelatin, sugars (including sucrose, glucose, dextrose, lactose and sorbitol), polyethylene glycol, waxes, natural and synthetic gums such as acacia, tragacanth, sodium alginate, cellulose, including hydroxypropylmethylcellulose (“HPMC”), microcrystalline cellulose (“MCC”), hydroxypropylcellulose, ethylcellulose, and veegum, and synthetic polymers such as acrylic acid and methacrylic acid copolymers, methacrylic acid copolymers, methyl methacrylate copolymers, aminoalkyl methacrylate copolymers, polyacrylic acid/polymethacrylic acid and polyvinylpyrrolidone (PVP).

Lubricants may facilitate tablet manufacture. Examples of suitable lubricants include, but are not limited to, magnesium stearate, calcium stearate, stearic acid, glycerol behenate, polyethylene glycol, talc, and mineral oil.

Disintegrants may facilitate dosage form disintegration after administration, and generally include, but are not limited to, starch, sodium starch glycolate, sodium carboxymethyl starch, sodium carboxymethylcellulose, hydroxypropyl cellulose, pregelatinized starch, clays, cellulose, alginine, gums or cross linked polymers, such as cross-linked PVP.

Stabilizers may inhibit or retard drug decomposition reactions which include, by way of example, oxidative reactions.

Surfactants may be anionic, cationic, amphoteric or nonionic surface active agents. Suitable anionic surfactants include, but are not limited to, those containing carboxylate, sulfonate and sulfate ions. Examples of anionic surfactants include sodium, potassium, ammonium of long chain alkyl sulfonates and alkyl aryl sulfonates such as sodium dodecylbenzene sulfonate; dialkyl sodium sulfosuccinates, such as sodium dodecylbenzene sulfonate; dialkyl sodium sulfosuccinates, such as sodium bis-(2-ethylthioxyl)-sulfosuccinate; and alkyl sulfates such as sodium lauryl sulfate. Cationic surfactants include, but are not limited to, quaternary ammonium compounds such as benzalkonium chloride, benzethonium chloride, cetrimonium bromide, stearyl dimethylbenzyl ammonium chloride, polyoxyethylene and coconut amine. Examples of nonionic surfactants include ethylene glycol monostearate, propylene glycol myristate, glyceryl monostearate, glyceryl stearate, polyglyceryl-4-oleate, sorbitan acylate, sucrose acylate, PEG-150 laurate, PEG-00 monolaurate, polyoxyethylene monolaurate, polysorbates, polyoxyethylene octylphenylether, PEG-1000 cetyl ether, polyoxyethylene tridecyl ether, polypropylene glycol butyl ether, stearoyl monoisopropanolamide, and polyoxyethylene hydrogenated tallow amide. Examples of amphoteric surfactants include sodium N-dodecyl-β-alanine, sodium N-lauryl-β-iminodipropionate, myristoamphoacetate, lauryl betaine and lauryl sulfobetaine.

If desired, the tablets, beads, granules, or particles may also contain minor amount of nontoxic auxiliary substances such as wetting or emulsifying agents, dyes, pH buffering agents, or preservatives.

### b) Topical Formulations and Patches

Pharmaceutical formulations adapted for transferal administration may be presented as discrete patches intended to remain in intimate contact with the epidermis of the recipient for a prolonged period of time. For example, the active ingredient may be delivered from the patch by iontophoresis as generally described in Pharmaceutical Research, 3(6), p 318 et seq. (1986).

Pharmaceutical formulations adapted for topical administration may be formulated as ointments, creams, suspensions, lotions, powders, solutions, pastes, gels, sprays, aerosols or oils.

For treatments of the eye or other external tissues, for example mouth and skin, the formulations are preferably applied as a topical ointment or cream.

When formulated in an ointment, the active ingredient may be employed with either a paraffinic or a water-miscible ointment base. Alternatively, the active ingredient may be formulated in a cream with an oil-in-water cream base or a water-in-oil base.

Pharmaceutical formulations adapted for topical administrations to the eye include eye drops wherein the active ingredient is dissolved or suspended in a suitable carrier, especially an aqueous solvent.

Pharmaceutical formulations adapted for topical administration in the mouth include lozenges, pastilles and mouth washes.

Pharmaceutical formulations adapted for rectal administration may be presented as suppositories or as enemas; rectal ointments and foams may also be employed.

Pharmaceutical formulations adapted for vaginal administration may be presented as pessaries, tampons, creams, gels, pastes, foams or spray formulations.

### c) Inhalable Formulations

Pharmaceutical formulations adapted for administration by inhalation include fine particle dusts or mists which may be generated by means of various types of metered dose pressurized aerosols, nebulizers or insufflators.

Spray compositions may, for example, be formulated as aerosols delivered from pressurized packs, such as a metered dose inhaler, with the use of a suitable liquified propellant.

Capsules and cartridges for use in an inhaler or insufflator, for example gelatine, may be formulated containing a powder mix for inhalation of a compound of the invention and a suitable powder base such as lactose or starch. Each capsule or cartridge may generally contain between about 1 μg and 10 mg of the compound of Structural Formula I, Structural Formula II, Structural Formula III or Structural Formula IV or combinations thereof.

Aerosol formulations are preferably arranged so that each metered dose or “puff” of aerosol contains about 1 μg to about 2000 μg, such as about 1 μg to about 500 μg of a compound of Structural Formula I, Structural Formula II, Structural Formula III or Structural Formula IV or combinations thereof. Administration may be once daily or several times daily, for example 2, 3, 4 or 8 times, giving for example 1, 2 or 3 doses each time. The overall daily dose with an aerosol will generally be within the range 10 μg to about 10 mg, such as 100 μg to about 2000 μg.

Pharmaceutical formulations adapted for nasal administration wherein the carrier is a solid include a coarse powder having a particle size for example in the range 20 to 500 microns which is administered in the manner in which snuff is taken, i.e. by rapid inhalation through the nasal passage from a container of the powder held close to the nose. Suitable formulations wherein the carrier is a liquid, for administration as a nasal spray or as nasal drops, include aqueous or oil solutions of the active ingredient.

The overall daily dose and the metered dose delivered by capsules and cartridges in an inhaler or insufflator will generally be double those with aerosol formulations.

### d) Injectable Formulations

Pharmaceutical formulations adapted for parenteral administration include aqueous and non-aqueous sterile injection solutions which may contain the antioxidants as well as buffers, bacteriostats and solutes which render the formulation isotonic with the blood of the intended recipient; and aqueous and non-aqueous sterile suspensions which may include suspending agents and thickening agents. The formulations may be presented in unit-dose or multi-dose containers, for example sealed ampules and vials, and may be stored in a freeze-dried (lyophilized) condition requiring only the addition of the sterile liquid carrier, for example water for injections, immediately prior to use. Extemporaneous injection solutions and suspensions may be prepared from sterile powders, granules and tablets.

Formulation of a metalloporphyrin derivative of the present invention in an intravenous lipid emulsion or a surfactant micelle or polymeric micelle (see., e.g., Jones et al., Eur. J. Pharmaceutics Biopharmaceutics 48, 101-111, 1999; Torchilin J. Clin, release 73, 137-172, 2001; both of which are incorporated herein by reference) is particularly preferred.

Sustained release injectable formulations are produced e.g., by encapsulating the metalloporphyrin derivative in porous microparticles which comprise a pharmaceutical agent and a matrix material having a volume average diameter between about 1 μm and 150 μm, e.g., between about 5 μm and 25 μm diameter. In one embodiment, the porous microparticles have an average porosity between about 5% and 90% by volume. In one embodiment, the porous microparticles further comprise one or more surfactants, such as a phospholipid. The microparticles may be dispersed in a pharmaceutically acceptable aqueous or non-aqueous vehicle for injection. Suitable matrix materials for such formulations comprise a biocompatible synthetic polymer, a lipid, a hydrophobic molecule, or a combination thereof. For example, the synthetic polymer can comprise, for example, a polymer selected from the group consisting of poly(hydroxy acids) such as poly(lactic acid), poly(glycolic acid), and poly(lactic acid-co-glycolic acid), poly(lactide), poly(glycolide), poly(lactide-co-glycolide), polyanhydrides, polyorthoesters, polyamides, polycarbonates, polyalkylenes such as polyethylene and polypropylene, polyalkylene glycols such as poly(ethylene glycol), polyalkylene oxides such as poly(ethylene oxide), polyalkylene terepthalates such as poly(ethylene terephthalate), polyvinyl alcohols, polyvinyl ethers, polyvinyl esters, polyvinyl halides such as poly(vinyl chloride), polyvinylpyrrolidone, polysiloxanes, poly(vinyl alcohols), poly(vinyl acetate), polystyrene, polyurethanes and co-polymers thereof, derivatized celluloses such as alkyl cellulose, hydroxyalkyl celluloses, cellulose ethers, cellulose esters, nitro celluloses, methyl cellulose, ethyl cellulose, hydroxypropyl cellulose, hydroxy-propyl methyl cellulose, hydroxybutyl methyl cellulose, cellulose acetate, cellulose propionate, cellulose acetate butyrate, cellulose acetate phthalate, carboxylethyl cellulose, cellulose triacetate, and cellulose sulphate sodium salt (jointly referred to herein as “syntheti c̅ celluloses”), polymers of acrylic acid, methacrylic acid or copolymers or derivatives thereof including esters, poly(methyl methacrylate), poly(ethyl methacrylate), poly(butylmethacrylate), poly(isobutyl methacrylate), poly(hexylmethacrylate), poly(isodecyl methacrylate), poly(lauryl methacrylate), poly(phenyl methacrylate), poly(methyl acrylate), poly(isopropyl acrylate), poly(isobutyl acrylate), and poly(octadecyl acrylate) (jointly referred to herein as “polyacrylic acids”), poly(butyric acid), poly(valeric acid), and poly(lactide-co-caprolactone), copolymers, derivatives and blends thereof. In a preferred embodiment, the synthetic polymer comprises a poly(lactic acid), a poly(glycolic acid), a poly(lactic-co-glycolic acid), or a poly(lactide-co-glycolide).

### Indications

The metalloporphyrin derivatives of the invention are useful in the treatment of a range of conditions associated with apoptosis. As will be apparent to the skilled artisan, the compounds per se of the present invention possess such utility by virtue of their ability to inhibit binding of a ligand to the benzodiapine receptor (and preferably by their ability to bind to the receptor). Without being bound by any theory or mode of action, the efficacy of the compounds of the present invention also resides in their ability to block, inhibit or reduce opening of the mPTP or otherwise prevent the efflux of calcium and/or cytochrome C that would lead to apoptosis. Thus, the present invention is not limited in scope by the nature of any disease to be treated other than a requirement for aetiology and/or progression of the disease to be associated with apoptosis, and/or for the severity of one or more disease symptoms to be associated with apoptosis.

Apoptosis-associated diseases for which the present invention is particularly useful in treating include neurodegenerative diseases e.g., diseases selected from the group consisting of Alzheimer's disease, dementia, Parkinson's disease, Lou Gehrig disease, motor neuron disease, Huntington's disease and multiple sclerosis. The treatment of Parkinson's Disease is preferred.

By virtue of their catalase, superoxide dismutase and peroxidase activities, the metalloporphyrin derivatives, especially EUK-450, EUK-451, EUK-452 or EUK-453, are also useful for reducing oxyradical- or reactive oxygen-induced damage to cells of an individual. For example, oxyradical or reactive oxygen-induced damage may result from a stroke, Alzheimer's disease, dementia, Parkinson's disease, Lou Gehrig disease, motor neuron disorders, Huntington's disease, cancer, multiple sclerosis, systemic lupus erythematosus, scleroderma, eczema, dermatitis, delayed type hypersensitivity, psoriasis, gingivitis, adult respiratory distress syndrome, septic shock, multiple organ failure, inflammatory diseases, asthma, allergic rhinitis, pneumonia, emphysema, chronic bronchitis, AIDS, inflammatory bowel disease, gastric ulcers, pancreatitis, transplantation rejection, atherosclerosis, hypertension, congestive heart failure, myocardial ischemic disorders, angioplasty, endocarditis, retinopathy of prematurity, cataract formation, uveitis, rheumatoid arthritis, oxygen toxicity, herpes simplex infection, burns, osteoarthritis, aging, etc.

The metalloporphyrin derivatives, especially EUK-450, EUK-451, EUK-452 or EUK-453, are also useful for treating free-radical associated diseases such as, for example: ischemic reperfusion injury, inflammatory diseases, systemic lupus erythematosus, myocardial infarction, stroke, traumatic hemorrhage, spinal cord trauma, Crohn's disease, autoimmune diseases (e.g., rheumatoid arthritis, diabetes), cataract formation, uveitis, emphysema, gastric ulcers, oxygen toxicity, neoplasia, radiation sickness, and other pathological states discussed above, such as toxemia and acute lung injury.

### Dosage and Administration

Selecting an administration regimen for a therapeutic composition depends on several factors, including the serum or tissue turnover rate of the entity, the level of symptoms, the immunogenicity of the entity, and the accessibility of the target cells in the biological matrix. Preferably, an administration regimen maximizes the amount of therapeutic compound delivered to the patient consistent with an acceptable level of side effects. Accordingly, the amount of composition delivered depends in part on the particular entity and the severity of the condition being treated.

A compound can be provided, for example, by continuous infusion, or by doses at intervals of, e.g., one day, one week, or 1-7 times per week. Doses of a composition may be provided intravenously, subcutaneously, topically, orally, nasally, rectally, intramuscular, intracerebrally, or by inhalation. A preferred dose protocol is one involving the maximal dose or dose frequency that avoids significant undesirable side effects. A total weekly dose depends on the type and activity of the compound being used. For example, such a dose is at least about 0.05 μg/kg body weight, or at least about 0.2 μg/kg, or at least about 0.5 μg/kg, or at least about 1 μg/kg, or at least about 10 μg/kg, or at least about 100 μg/kg, or at least about 0.2 mg/kg, or at least about 1.0 mg/kg, or at least about 2.0 mg/kg, or at least about 10 mg/kg, or at least about 25 mg/kg, or at least about 50 mg/kg (see, e.g., Yang, et al. New Engl. J. Med. 349:427-434, or Herold, et al. New Engl. J. Med. 346:1692-1698, 2002.

An effective amount of a compound for a particular patient may vary depending on factors such as the condition being treated, the overall health of the patient, the method route and dose of administration and the severity of side affects, see, e.g., Maynard, et al. (1996) A Handbook of SOPs for Good Clinical Practice, Interpharm Press, Boca Raton, Fla.; or Dent (2001) Good Laboratory and Good Clinical Practice, Urch Publ., London, UK.

Determination of the appropriate dose is made by a clinician, e.g., using parameters or factors known or suspected in the art to affect treatment or predicted to affect treatment. Generally, the dose begins with an amount somewhat less than the optimum dose and is increased by small increments thereafter until the desired or optimum effect is achieved relative to any negative side effects. Important diagnostic measures include those of symptoms of the disease and/or disorder being treated. Preferably, a compound that will be used is derived from or adapted for use in the same species as the subject targeted for treatment, thereby minimizing a humoral response to the reagent.

An effective amount of therapeutic will decrease disease symptoms, for example, as described supra, typically by at least about 10%; usually by at least about 20%; preferably at least about 30%; more preferably at least about 40%, and more preferably by at least about 50%.

The route of administration is preferably by, e.g., topical or cutaneous application, injection or infusion by intravenous, intraperitoneal, intracerebral, intramuscular, intraocular, intraarterial, intracerebrospinal, intralesional, or pulmonary routes, or by sustained release systems or an implant (see, e.g., Sidman et al. Biopolymers 22:547-556, 1983; Langer, et al. J. Biomed. Mater. Res. 15:167-277, 1981; Langer Chem. Tech. 12:98-105, 1982; Epstein, et Proc. Natl. Acad. Sci. USA 82:3688-3692, 1985; Hwang, et al. Proc. Natl. Acad. Sci. USA 77:4030-4034, 1980; U.S. Pat. Nos. 6,350,466 and 6,316,024).

The pharmaceutical formulation of the present invention will generally contain sufficient porphyrin compound to reduce, delay or inhibit apoptosis of cells. This is determined by ant art-recognized means e.g., by determining apoptosis or cell lysis in the presence of the compound and a second compound known to induce or promote apoptosis. Opening of a mitochondrial permeability transition pore (mPTP) in a cell can also be determined as a measure of efficacy of the compound and/or effective dose of the compound. Mitochondrial membrane depolarization can also be determined. Alternatively, or in addition, the release of calcium and/or cytochrome C from a cell or mitochondrion can be determined.

The present invention is further described by reference to the following non-limiting examples.

### Example 1

### Syntheses of Compounds

In the following synthesis examples, all used chemicals should be of reagent grade. Column chromatography is carried out on silica gel 60 AC.C (6-35 μm), or basic alumina 90 (70-230 mesh). Analyses are carried out using one or more combinations of ¹H-NMR, TLC, UV-vis, HPLC and ESI-MS. Nuclear magnetic resonance spectra are recorded on a Bruker AMX 300 or AM 250 A or a Bruker AC 200 spectrometer. UV-visible spectra are obtained on Hewlett Packard 8452A diode array spectrophotometer. The mass spectra are recorded on a Nermag R10-10H for the FAB+ spectra and on a API 365 PE SCIEX for the electrospray spectra. Infrared spectra are recorded on a Perkin-Elmer 1725×FT-IR Spectrometer.

### 1. Dipyrromethane

Dipyrromethane was prepared according to the Lindsey method (Littler et al., J. Org. Chem. 64, 1391-1396, 1999, and essentially as described in International Patent Application No. PCT/US04/17560.

### 2. {[{(Porphine-5,15-diyl)bis[cyclopropyl-diyl]}](2-)-N²¹,N²²,N²³,N²⁴} manganese (III) acetate (EUK-418)

{21H,23H-porphine-5,15-diyl)bis[cyclopropyl-diyl]} was prepared from dipyrromethane and cyclopropanecarboxaldehyde essentially as described in International Patent Application No. PCT/US04/17560. This reaction involves the condensation of dipyrromethane and aldehyde under high-dilution conditions using trifluoroacetic acid as a catalyst, and oxidization with 2,3-dichloro 5,6-dicyanobenzoquinone (DDQ). The porphyrin was characterized by ¹H-NMR and TLC.

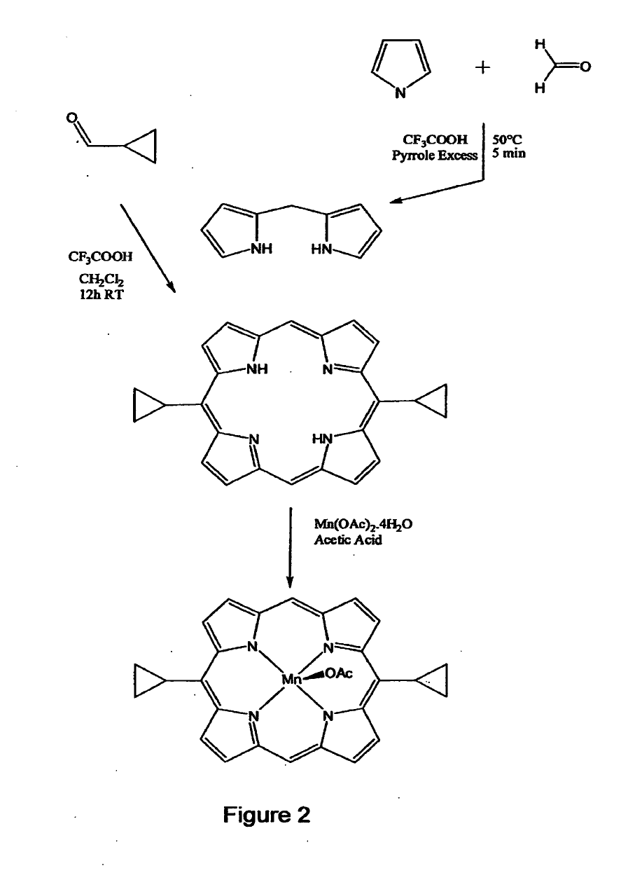

In one reaction scheme, EUK-418 is prepared from {21H,23H-porphine-5,15-diyl)bis[cyclopropyl-diyl]} in dimethyl formamide (DMF) by reaction with 2,4,6-collidine and Mn(OAc)₂.4H₂O essentially as described in International Patent Application No. PCT/US04/17560. This reaction scheme is shown in FIG. 2.

In an alternative reaction scheme, manganese was incorporated into {21H,23H-porphine-5,15-diyl)bis[cyclopropyl-diyl]} using standard conditions. In particular, Mn(OAc)₂.4H₂O was added to the free-base porphyrin in acetic acid and the mixture heated over several hours monitoring the progress of the reaction by UV-vis light. The compound was worked up under neutral conditions. Yields by this route were typically greater than 85%. The purity and identity of the EUK-418 derivative was assessed by TLC, UV-vis, ESI-MS, and HPLC.

### 3. {[{(Porphine-5,15-diyl)bis[benzyl-diyl]}](2-)-N²¹,N²²,N²³,N²⁴} manganese (III) acetate (EUK-423)

{(21H,23H-Porphine-5,15-diyl)bis[benzyl-diyl]} was prepared according to the method described by Manka and Lawrence, Tetrahedron Letters, 30, 6989-6992, 1989, from dipyrromethane and benzaldehyde. This reaction involves the condensation of dipyrromethane and benzaldehyde under high-dilution conditions using trifluoroacetic acid as a catalyst, and oxidization with 2,3-dichloro 5,6-dicyanobenzoquinone (DDQ). The porphyrin was characterized by ¹H-NMR and TLC.

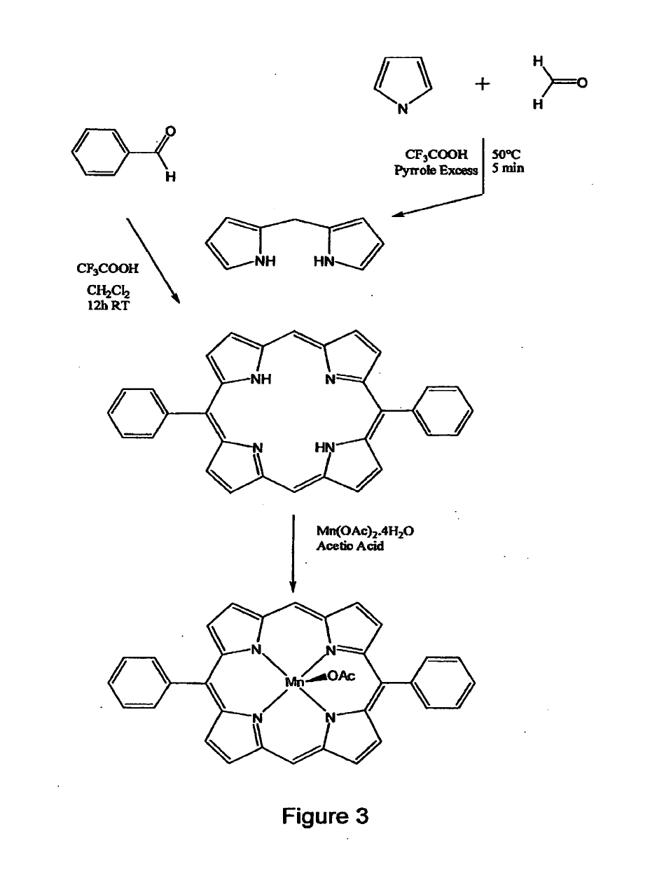

In one reaction scheme, EUK-423 is prepared from {(21H,23H-Porphine-5,15-diyl)bis[benzyl-diyl]} in dimethyl formamide (DMF) by reaction with 2,4,6-collidine and Mn(OAc)₂.4H₂O essentially as described in International Patent Application No. PCT/US04/17560. This reaction scheme is shown in FIG. 3.

In an alternative reaction scheme, manganese was incorporated into {(21H,23H-Porphine-5,15-diyl)bis[benzyl-diyl]} by addition of Mn(OAc)₂.4H₂O in acetic acid, and then heating the mixture over several hours, monitoring the progress of the reaction by UV-vis light. The compound was worked up under neutral conditions. Yields by this route were typically greater than 85%. The purity and identity of the EUK-423 derivative was assessed by TLC, UV-vis, ESI-MS, and HPLC.

### 4. (5,10,15,20-Tetraisopropylporphyrinato)manganese(III)acetate(EUK-424)

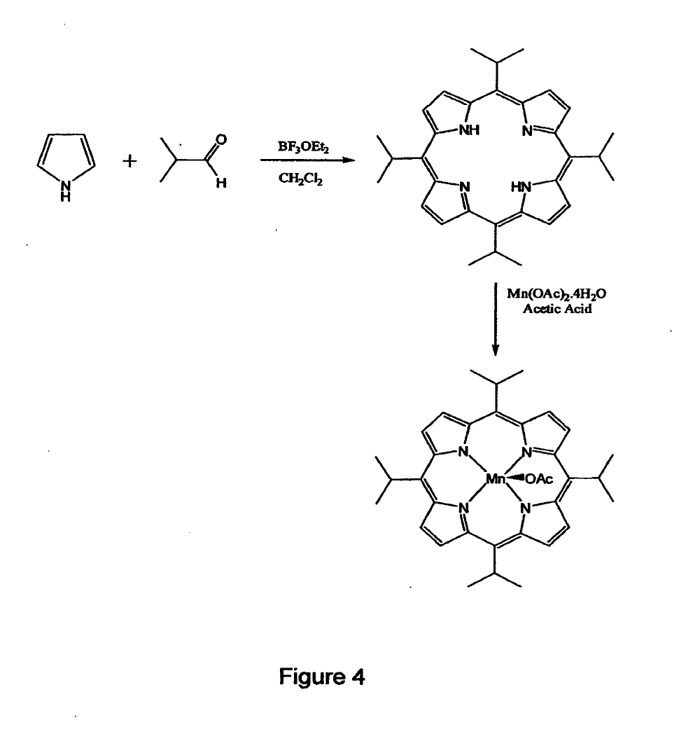

5,10,15,20-Tetraisopropylporphyrin was prepared according to the method described by Senge et al., J. Porphyrins and Phthalocyanines 3, 99-116, 1999 (FIG. 4). The porphyrin was characterized by ¹H-NMR and TLC.

In one reaction scheme, EUK-418 is prepared from 5,10,15,20-Tetraisopropylporphyrin in dimethyl formamide (DMF) by reaction with 2,4,6-collidine and Mn(OAc)₂.4H₂O essentially as described in International Patent Application No. PCT/US04/17560. This reaction scheme is shown in FIG. 4.

In an alternative reaction scheme, manganese was incorporated into 5,10,15,20-Tetraisopropylporphyrin by addition of Mn(OAc)₂.4H₂O in acetic acid, and then heating the mixture over several hours, monitoring the progress of the reaction by UV-vis light. The compound was worked up under neutral conditions. Yields by this route were typically greater than 85%. The purity and identity of the EUK-424 derivative was assessed by TLC, UV-vis, ESI-MS, and HPLC.

### 5. (5,10,15,20-Tetraethylporphyrinato)manganese (III) acetate (EUK-425)

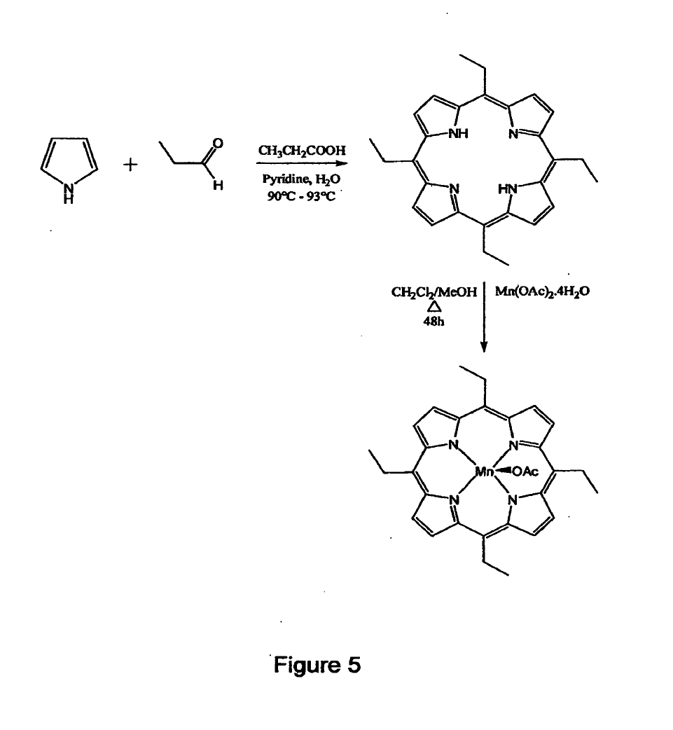

5,10,15,20-Tetraethylporphyrin was prepared as described by Neya et al., J. Heterocyclic Chem., 34, 689-690, 1997 (FIG. 5). The porphyrin was characterized by ¹H-NMR and TLC.

To produce (5,10,15,20-Tetraethylporphyrinato)manganese(III)acetate (EUK-425), a solution of 0.58 g (2.3 mmol) of Mn(OAc)₂.4H₂O in 50 ml of methanol was added to a solution of 0.05 g (0.12 mmol) of 5,10,15,20-Tetraethylporphyrin in 100 ml of CH₂Cl₂. The reaction mixture under nitrogen was heated 48 h under reflux. Then 100 ml H₂O were added to the cooled solution and metallated porphyrin was extracted with 200 ml of CH₂Cl₂. The organic layer was dried over sodium sulphate and filtered. Solvents were removed under vacuum and the crude product was dissolved in a minimum quantity of CH₂Cl₂. A large amount of n-hexane was then added until a precipitate is obtained. The precipitate was filtered off, washed several times with n-hexane leading to a dark powder comprising (5,10,15,20-Tetraethylporphyrinato)manganese(III) acetate (EUK-425). Yields were typically about 36%. The method is essentially as described in International Patent Application No. PCT/US04/17560. This reaction scheme is shown in FIG. 5.

### 6. (5,10,15,20-Tetramethylporphyrinato)manganese(III) acetate (EUK-426)

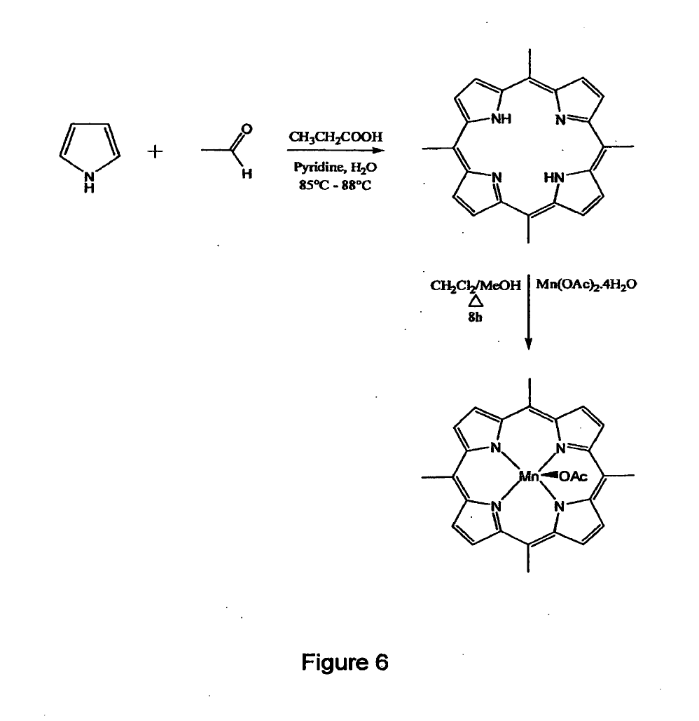

5,10,15,20-Tetramethylporphyrin was prepared as described by Neya et al., J. Heterocyclic Chem., 34, 689-690, 1997 (FIG. 6). The porphyrin was characterized by ¹H-NMR and TLC.

To produce (5,10,15,20-Tetramethylporphyrinato)manganese(III)acetate (EUK-426), a solution of 0.67 g (2.7 mmol) of Mn(OAc)₂.4H₂O in 50 ml of methanol is added to a solution of 0.05 g (0.13 mmol) of 5,10,15,20-Tetramethylporphyrin in 100 ml CH.sub.2Cl.sub.2. The reaction mixture is heated 8 hr under reflux and nitrogen. Then 100 ml H₂O are added to the cooled solution and metallated porphyrin is extracted with 200 ml CH₂Cl₂. The organic layer is dried over sodium sulphate and filtered. Solvents are removed under vacuum and the crude product is dissolved in a minimum quantity of CH₂Cl₂. A large amount of n-hexane is then added until a precipitate is obtained. The precipitate is filtered off, washed several times with n-hexane leading to a dark powder comprising EUK-426. Yields are typically about 60%. The method is essentially as described in International Patent Application No. PCT/US04/17560. This reaction scheme is shown in FIG. 6.

In an alternative reaction scheme, manganese was incorporated into 5,10,15,20-Tetramethylporphyrin by addition of Mn(OAc)₂.4H₂O in acetic acid, and then heating the mixture over several hours, monitoring the progress of the reaction by UV-vis light. The compound was worked up under neutral conditions. Yields by this route were typically greater than 85%. The purity and identity of the EUK-426 derivative was assessed by TLC, UV-vis, ESI-MS, and HPLC.

### 7. {[{(Porphine-5,15-diyl)bis[benzene-1,4 diyl(4-methyl-oxy)]}](2-)-N²¹,N²², N²³,N²⁴} manganese(III) acetate (EUK-450)

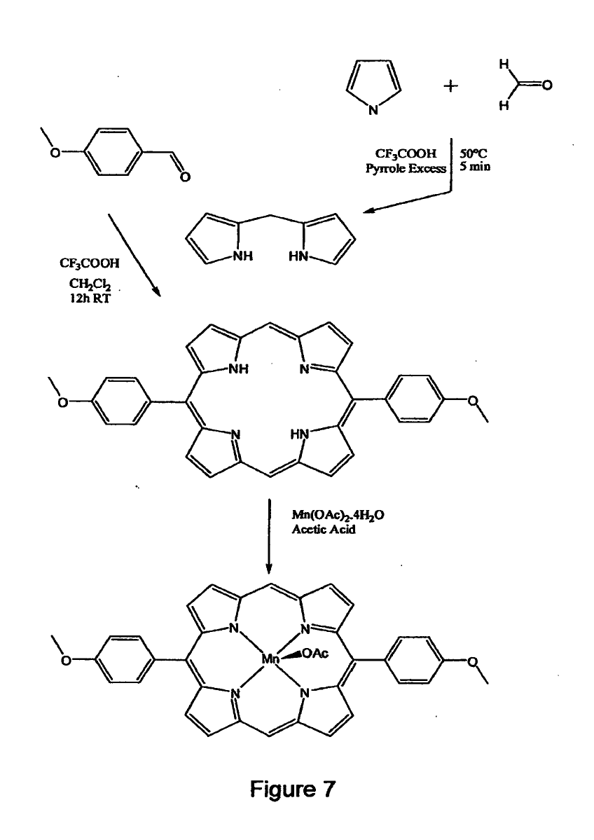

{(21H,23H-Porphine-5,15-diyl)bis[benzene-1,4 diyl(4-methyl-oxy)]} was prepared essentially as shown in FIG. 7. This reaction involves the condensation of dipyrromethane and aldehyde under high-dilution conditions using trifluoroacetic acid as a catalyst, and oxidization with 2,3-dichloro 5,6-dicyanobenzoquinone (DDQ). The porphyrin was characterized by ¹H-NMR and TLC.

In one reaction scheme, EUK-450 is prepared from {(21H,23H-Porphine-5,15-diyl)bis[benzene-1,4 diyl(4-methyl-oxy)]} in dimethyl formamide (DMF) by reaction with 2,4,6-collidine and Mn(OAc)₂.4H₂O essentially as described herein for EUK-418. This reaction scheme is shown in FIG. 7.

In an alternative reaction scheme, manganese was incorporated into {(21H,23H-Porphine-5,15-diyl)bis[benzene-1,4 diyl(4-methyl-oxy)]} by addition of Mn(OAc)₂.4H₂O to the free-base porphyrin in acetic acid and heating the mixture for several hours, monitoring the progress of the reaction by UV-vis light. The compound was worked up under neutral conditions. Yields by this route were typically greater than 85%. The purity and identity of the EUK-450 derivative was assessed by TLC, UV-vis, ESI-MS, and HPLC.

### 8. {[{(Porphine-5,15-diyl)bis[4-Tetrahydropyrano-diyl]}](2-)-N²¹,N²²,N²³,N²⁴} manganese(III) chloride (EUK-451)

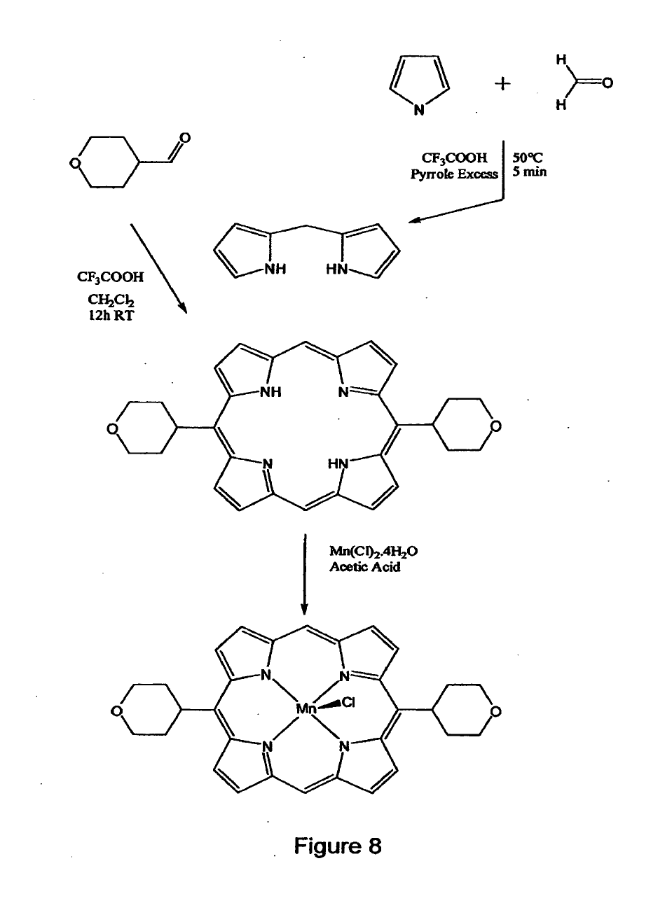

{(21H,23H-Porphine-5,15-diyl)bis[4-Tetrahydropyrano-diyl]} was prepared essentially as shown in FIG. 8. This reaction involves the condensation of dipyrromethane and aldehyde under high-dilution conditions using trifluoroacetic acid as a catalyst, and oxidization with 2,3-dichloro 5,6-dicyanobenzoquinone (DDQ). The porphyrin was characterized by ¹H-NMR and TLC.

In one reaction scheme, EUK-451 is prepared {(21H,23H-Porphine-5,15-diyl)bis[4-Tetrahydropyrano-diyl]} in dimethyl formamide (DMF) by reaction with 2,4,6-collidine and Mn(Cl)₂.4H₂O essentially as shown in FIG. 8.

In an alternative reaction scheme, manganese was incorporated into {(21H,23H-Porphine-5,15-diyl)bis[4-Tetrahydropyrano-diyl]} by addition of Mn(Cl)₂.4H₂O to the free-base porphyrin in acetic acid and heating the mixture for several hours, monitoring the progress of the reaction by UV-vis light. The compound was worked up by washing the organic phase with 1N HCl. Yields by this route were typically greater than 85%. The purity and identity of the EUK-451 derivative was assessed by TLC, UV-vis, ESI-MS, and HPLC.

### 9. {[{(Porphine-5,15-diyl)bis[cyclohexyl-diyl]}](2-)-N²¹,N²²,N²³,N²⁴} manganese(III) chloride (EUK-452)

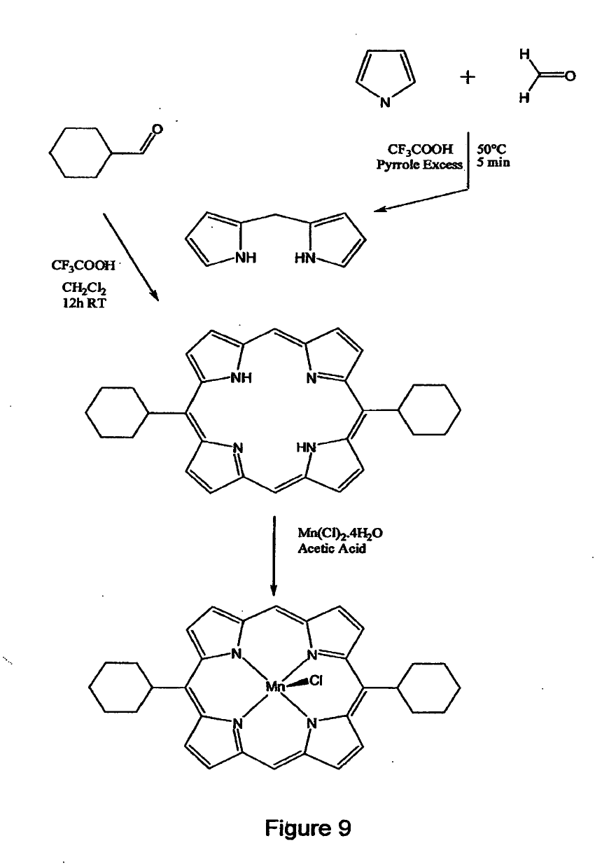

{(21H,23H-Porphine-5,15-diyl)bis[cyclohexyl-diyl]} was prepared essentially as shown in FIG. 9. This reaction involves the condensation of dipyrromethane and aldehyde under high-dilution conditions using trifluoroacetic acid as a catalyst, and oxidization with 2,3-dichloro 5,6-dicyanobenzoquinone (DDQ). The porphyrin was characterized by ¹H-NMR and TLC.

In one reaction scheme, EUK-452 is prepared {(21H,23H-Porphine-5,15-diyl)bis[cyclohexyl-diyl]} in dimethyl formamide (DMF) by reaction with 2,4,6-collidine and Mn(Cl)₂.4H₂O essentially as shown in FIG. 9.

In an alternative reaction scheme, manganese was incorporated into {(21H,23H-Porphine-5,15-diyl)bis[cyclohexyl-diyl]} by addition of Mn(Cl)₂.4H₂O to the free-base porphyrin in acetic acid and heating the mixture for several hours, monitoring the progress of the reaction by UV-vis light. The compound was worked up by washing the organic phase with 1N HCl. Yields by this route were typically greater than 85%. The purity and identity of the EUK-452 derivative was assessed by TLC, UV-vis, ESI-MS, and HPLC.

### 10. {[{(Porphine-5,15-diyl)bis[propyl-diyl]}](2-)-N²¹,N²²,N²³,N²⁴} manganese (III) chloride (EUK-453)

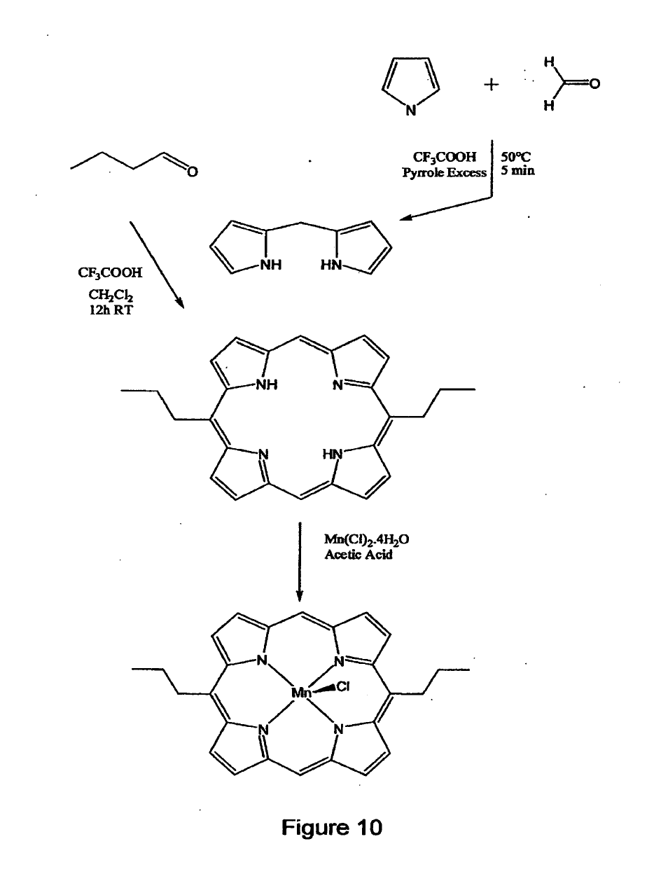

{(21H,23H-Porphine-5,15-diyl)bis[propyl-diyl]} was prepared essentially as shown in FIG. 10. This reaction involves the condensation of dipyrromethane and n-butaldehyde under high-dilution conditions using trifluoroacetic acid as a catalyst, and oxidization with 2,3-dichloro 5,6-dicyanobenzoquinone (DDQ). The porphyrin was characterized by ¹H-NMR and TLC.

In one reaction scheme, EUK-453 is prepared {(21H,23H-Porphine-5,15-diyl)bis[propyl-diyl]} in dimethyl formamide (DMF) by reaction with 2,4,6-collidine and Mn(Cl)₂.4H₂O essentially as shown in FIG. 10.

In an alternative reaction scheme, manganese was incorporated into {(21H,23H-Porphine-5,15-diyl)bis[propyl-diyl]} by addition of Mn(Cl)₂.4H₂O to the free-base porphyrin in acetic acid and heating the mixture for several hours, monitoring the progress of the reaction by UV-vis light. The compound was worked up by washing the organic phase with 1N HCl. Yields by this route were typically greater than 85%. The purity and identity of the EUK-452 derivative was assessed by TLC, UV-vis, ESI-MS, and HPLC.

### Example 2

### Protection Against STS-Induced Apoptosis in PC12 Cells

### Methods

Rat pheochromocytoma (PC12) cells were cultured in collagen-coated 96-well plates according to directions provided by the American Type Culture Collection. Staurosporine was added at various concentrations sufficient to induce apoptosis. Test compounds were added together with the staurosporine. Cells were incubated overnight at 37° C., 5% CO₂. After 18-24 hours, test media was removed and cell viability was determined using the XTT viability assay described by Baker et al., J. Pharmacol. Exp, Therapeutics 284, 215-221, 1998, the contents of which are incorporated herein by reference.

### Results

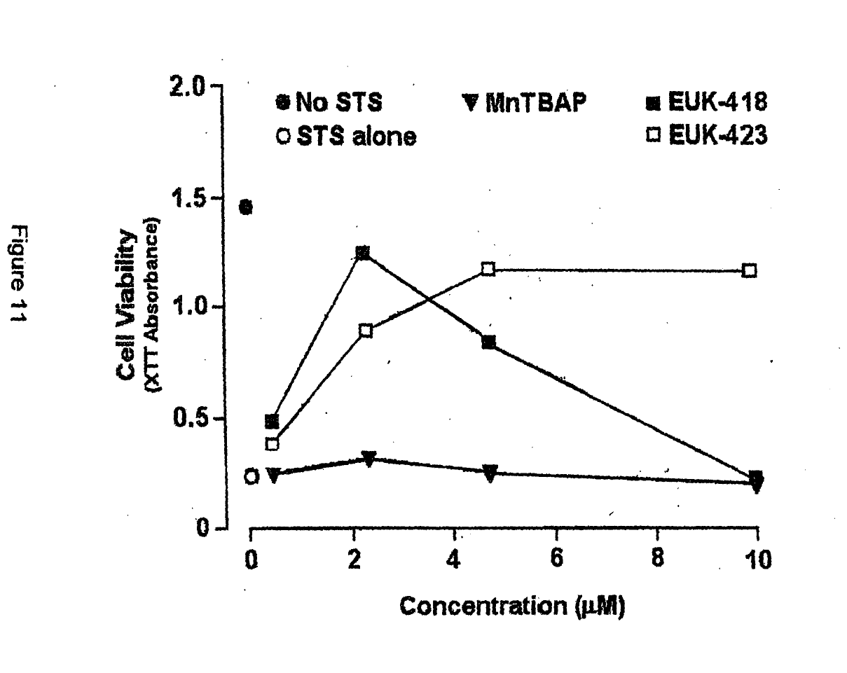

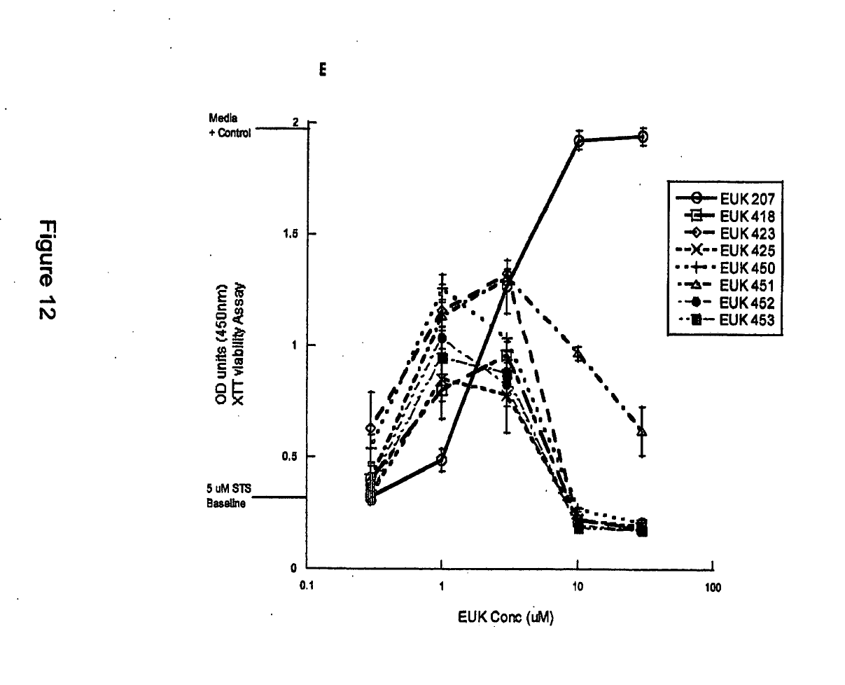

Data showing a protective effect conferred by the compounds of the present invention in separate experiments are provided in FIGS. 11 and 12. Data indicate that all compounds provide protection against STS-induced apoptosis of PC12 cells at low concentration i.e., in the range up to about 3-5 μM. EUK-451 shows the highest activity and the lowest toxicity (FIG. 12).

### Example 3

### Protection Against Radiation-Induced Apoptosis

### Methods

Bovine capillary endothelial cells were cultured on eight-chamber Labtek slides and exposed to ionizing radiation (20 Gy), which was calibrated using an X-ray exposure meter. The compounds designated EUK-418, EUK-423, EUK-425, EUK-450, EUK-451, and EUK-452 (in the range of 0.5 μM to 100 μM concentration) were added to cultures immediately after irradiation. In control experiments, cells either received no ionizing radiation (sham) or no compound. After 6 h incubation, the cells were fixed in methanol and stained with 5 μg/ml 4,6-diamidino-2-phenylindole (DAPI). DNA was visualized using a Nikon epifluorescence microscope, and apoptosis was scored and expressed as an apoptotic index (% apoptotic cells in a field of 100). Because necrosis was observed at the doses tested for EUK-425, the data were expressed as field number i.e., the number of fields necessary to count 100 cells. Field number was also calculated for all other compounds tested as a control (results not shown).

To determine cytotoxicity of the compounds under these conditions, duplicate samples of the cells receiving a dose of compound at each concentration tested in the absence of a dose of ionizing radiation were assayed for release of LDH into the culture medium.

### Results

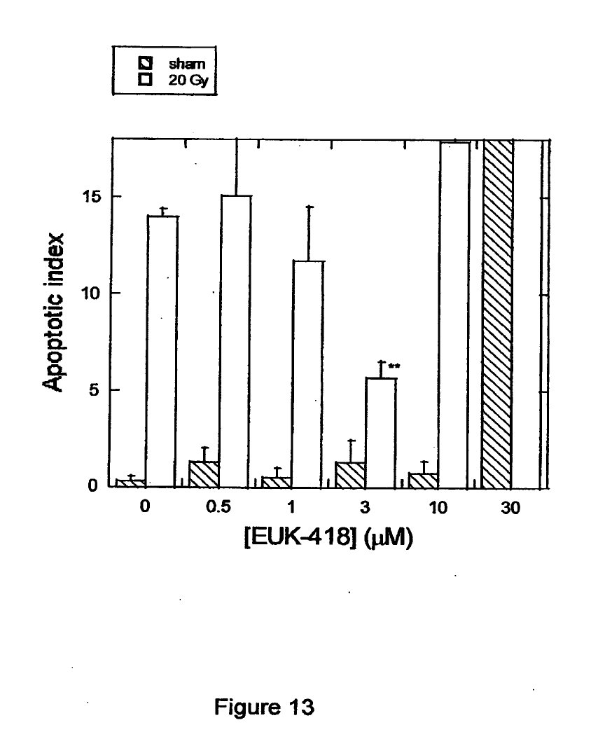

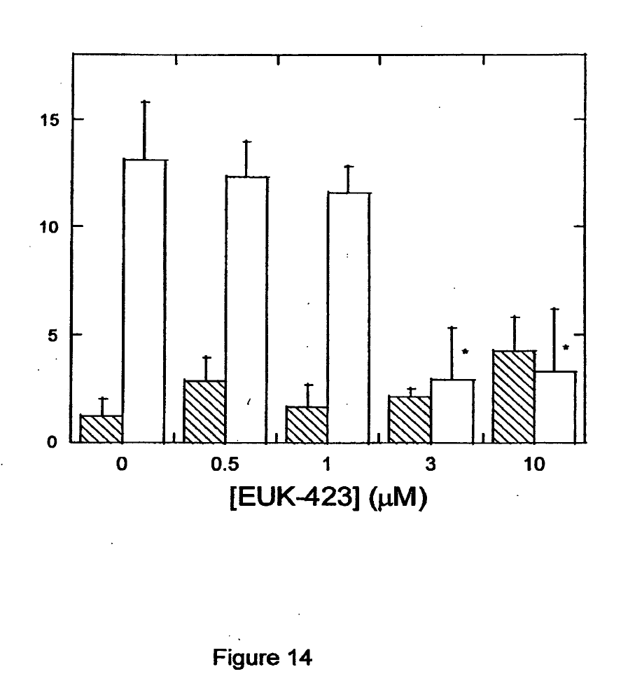

Data presented in FIG. 13 and FIG. 14 demonstrate that low doses of EUK-418 and EUK-423 protected bovine capillary endothelial cells exposed to ionizing radiation.

For example, 3 μM EUK-418 provided significant protection (p<0.001) against the effect of such ionizing radiation (FIG. 13). Under these conditions, the compound failed to induce noticeable necrosis of bovine capillary endothelial cells. In contrast, only 30 μM EUK-418 or higher caused significant increase in field number for control cells, indicating cytotoxicity.

Concentrations of about 3-10 μM EUK-423 conferred approximately 87% protection on bovine capillary endothelial cells exposed to this dose if ionizing radiation (FIG. 14). The effect was significant at p<0.006. Under these conditions, there was no detectable necrosis of cells from cytoxicity for the compounds (FIG. 14). In contrast, only 30 μM EUK-423 or higher caused significant increase in field number for control cells, indicating cytotoxicity.

Other compounds of the invention were also tested and shown to confer protection against radiation-induced apoptosis under these conditions (data not shown). In particular, the following concentrations of compounds were protective: EUK-425 (1 μM and 3 μM), EUK-450 (1 μM and 3 μM), EUK-451 (1 μM and 3 μM), and EUK-452 (3 μM). Assays of LDH release in the presence of these compounds indicated that they are not significantly toxic at concentrations which are protective against this dose of ionizing radiation. As with EUK-418 and EUK-423, most of the compounds are cytotoxic at about 30 μM concentration of higher, with the notable exception of EUK-451 which induced LDH release only at 100 μM concentration of higher.

### Example 4

### Inhibition of PK11195 Binding of Compounds to the Diazapine Receptor

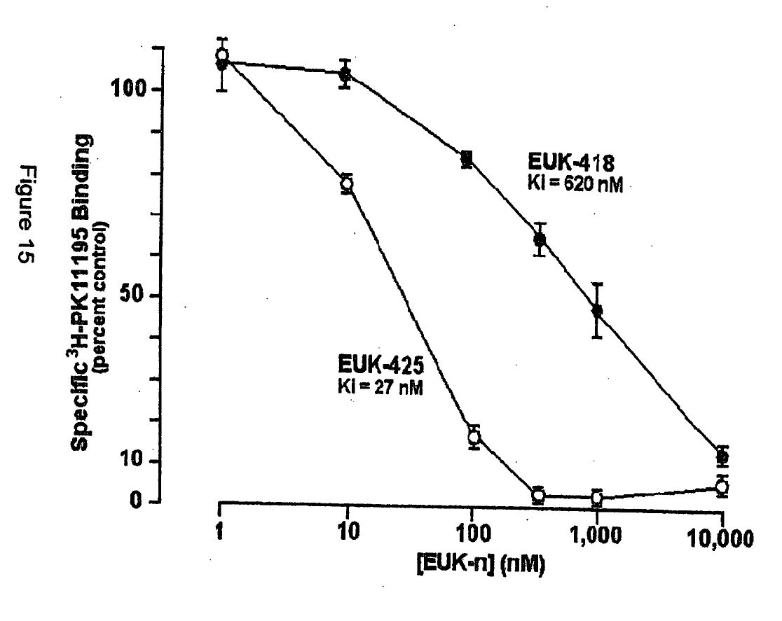

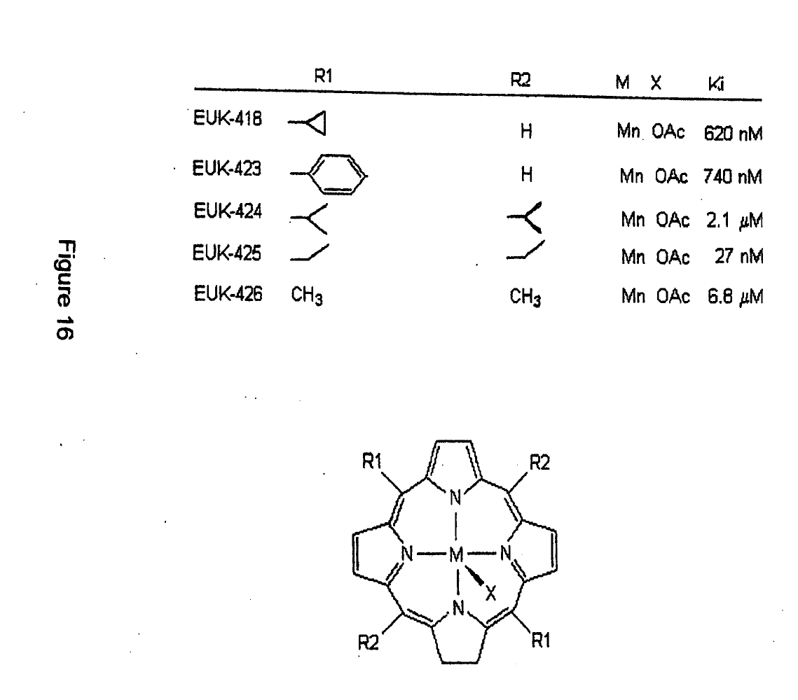

Data presented in FIG. 15 and FIG. 16 indicate that the compounds EUK-418, EUK-423, EUK-424, EUK-425 and EUK-426 inhibit binding of a ligand of the mitochondrial benzodiazepine receptor with moderate affinity. One of the compounds having the highest affinity for inhibition, EUK-425, displays a Ki of 27 nM against the binding of PK11195, a standard ligand for the BZD receptor (Le Fur et al., Life Sci 32(16), 1849-1856, 1983; Le Fur et al., Life Sci 32(16), 1839-1847, 1983; and Le Fur et al., Life Sci 33(5), 449-457, 1983; all of which are incorporated herein by reference).

While binding to the mPTP can lead to its opening, and subsequent cellular death through apoptosis, it can also lead to prevention of that pathway, and, whilst not being bound by any theory or mode of action, the compounds of the invention may bind to the mPTP to inhibit, prevent or delay its opening.

### Example 5

### Efficacy of Compounds in a Model of Parkinson's Disease In Vitro

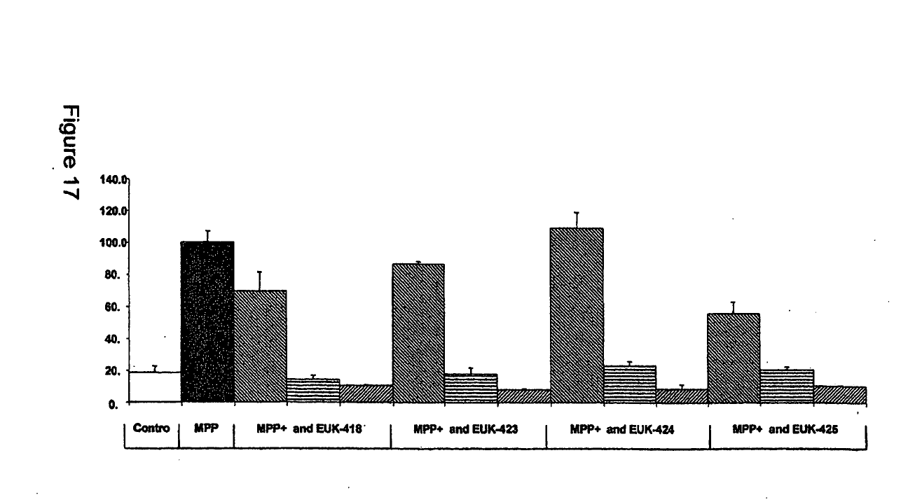

Data presented in FIG. 17 indicate that EUK-418, EUK-423, EUK-424 and EUK-425 prevent cytotoxicity induced by MPP+ in cultured slices from mesencephalon, an in vitro model for Parkinson's disease. For example, the EC₅₀ of EUK-425 in this model was less than 100 nM, correlating with the affinity of the compound for the mPTP.

### Example 6

### Oral Bioavailability of Compounds

### A) Oral Bioavailability In Vitro

### Methods

To measure stability of the compounds in USP simulated gastric fluid (SGF; i.e., 34 mM NaCl, 3.2 mg/ml pepsin, 81.2 mM HCl, pH approx. 1.2), compounds designated EUK-418, EUK-423, EUK-425, EUK-450, EUK-452, EUK-452 and EUK-453 were diluted in SGF and incubated at 37° C. for one hour or longer. Aliquots of SGF were withdrawn and intact compound quantitated by HPLC-UV.

To indicate the likelihood that the compounds will cross lipid membranes and be absorbed once administered by oral means, their lipophilicities were determined by octanol partitioning. Octanol partitioning coefficients were determined by standard methods using octanol-water mixtures as described previously (Melov et al., J. Neuroscience 21, 8348-8353, 2001). Quantitation was performed by HPLC-UV.

### Results

All compounds tested were stable to incubation in simulated gastric fluid at 37° C. for 90 minutes (Table 1). This time period is longer than the standard gastric transit time of 60 mins advised by the U.S. Food and Drug Administration (FDA). These data thus suggest that degradation of compounds in the acid environment of the stomach is not a barrier to their oral availability.

TABLE 1
Stability of compounds in SGF

| | Percentage remaining |
|---|---|
| Compound Designation | after 90 mins in SGF |
| EUK-418 | 92.2% |
| EUK-423 | 91.2% |
| EUK-425 | 97.0% |
| EUK-450 | 98.1% |
| EUK-451 | 100% |
| EUK-452 | 98.2% |
| EUK-453 | 98.4% |

Octanol partitioning coefficient (P) was determined for each compound as a measure of its lipophilicity, which is predictive of their ability to cross membrane barriers such as the blood-brain barrier (Table 2). Classically, a log₁₀ P value of 2.0 or greater is predictive of an ability to cross the blood-brain barrier (Hansch et al., 1987). With the exception of EUK-451, all of the compounds tested are substantially lipophilic, suggesting that their ability to be absorbed into endothelial membranes is not a significant barrier to their oral bioavailability. EUK-452 was the most lipophilic, with a log₁₀ P value of 1.98.

TABLE 2
Lipophilicity of compounds as determined
by octanol partitioning coefficient (P)

| Compound Designation | P value | Log₁₀ P value |
|---|---|---|
| EUK-418 | 3.53 ± 0.14 | 0.548 ± 0.009 |
| EUK-423 | 8.07 ± 0.10 | 0.907 ± 0.002 |
| EUK-425 | 8.56 ± 0.35 | 0.932 ± 0.006 |
| EUK-450 | 4.46 ± 0.11 | 0.650 ± 0.010 |
| EUK-451 | 0.219 ± 0.005 | −0.660 ± 0.011 |
| EUK-452 | 93.97 ± 1.67 | 1.973 ± 0.008 |
| EUK-453 | 4.16 ± 0.16 | 0.619 ± 0.017 |

Another commonly used in vitro model for oral availability is permeability through a Caco-2 monolayer (Gres et al., Pharm Res. 15, 726-733, 1998; Stoner et al., Int. J. Pharm. 269, 241-249, 2004; and Yee, Pharm Res. 14, 763-766, 1997). This model system is not useful for the compounds described herein, because they became associated with the cellular layer.

### B) Oral Bioavailability In Vivo

### Methods

In this study, oral bioavailability of both EUK-418 and EUK-423 was demonstrated in rats. Fasted and fed Sprague-Dawley rats were dosed by intragastric gavage with either 4 mg/kg EUK-418 or 2 mg/kg EUK-423. At time points up to 7 hr post-administration, animals were sacrificed, their blood collected over heparin, plasma samples prepared by centrifugation, and plasma levels of the compounds were determined by LC-MS/MS.

Oral bioavailability of each compound is determined by comparing plasma levels of the compound after administration at all time points, and by comparing the AUC (area under the curve) calculated from these data.

### Results

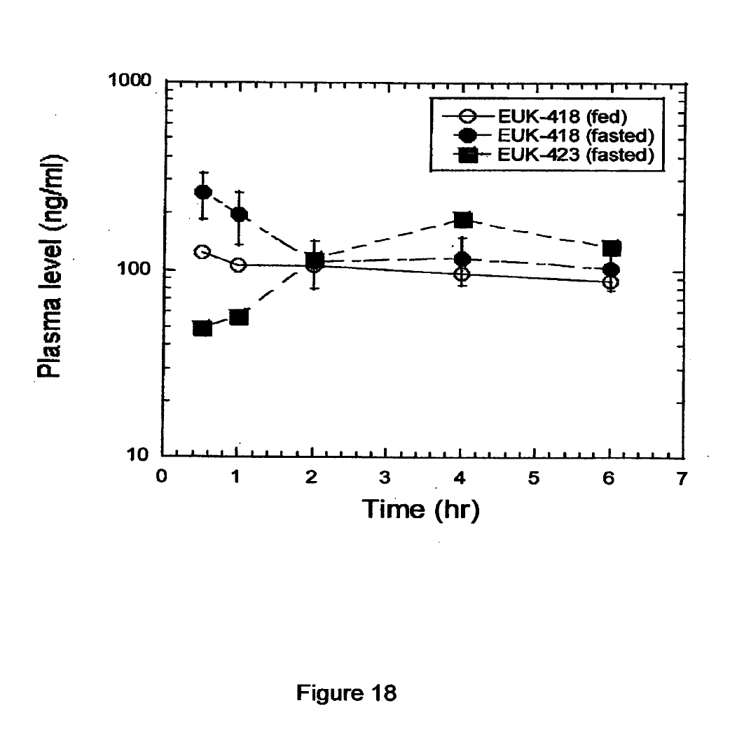

Data presented in FIG. 18 indicate that both EUK-418 and EUK-423 are found in plasma following oral administration by gastric gavage and, as a consequence are bioavailable in vivo. EUK-423 increased in plasma during the first four hour period following intragastric gavage with the compound, whereas EUK-418 increased rapidly in serum of fasted rats and then declined to about 100 ng/ml concentration for the assayed period. Fasting of animals also appeared to increase plasma concentration of EUK-418. Under similar conditions, two control compounds were shown to be undetectable in plasma (data not shown).

In another study, a 3.5 mg/kg dose of EUK-451 administered orally was also recovered from plasma at low levels (not shown).

These data are consistent with predictions of in vitro bioavailability (Table 1; Table 2), suggesting that octanol partitioning and SGF stability are useful predictors of oral availability for these types of compounds. By extrapolation based upon the stability and octanol partitioning data presented herein, the compounds tested, with the possible (albeit not certain) exception of EUK-452 which is much more lipophilic than either EUK-418 or EUK-423, are predicted to be bioavailable in vivo.

### C) Ability of Compounds to Cross Blood-Brain Barrier

In further studies, compounds are administered orally to animals as described above, or alternatively, by daily i.p. injection for up to five days at concentrations of about 0.3 mg/kg body weight, 3.0 mg/kg and 30 mg/kg. The animals are then sacrificed and their brains removed and snap-frozen in liquid nitrogen. The brains are homogenized in methanol containing 5% TCA, and the soluble fraction is prepared by centrifugation.

Levels of compounds in the brain tissue are determined by LC-MS (HPLLC, Waters; Mass Spectrometer, Micromass Quattro). The half-life of each compound administered to the animals is determined over a period of time from about 5 minutes up to about 2 days.

Brain bioavailability is determined by calculating brain uptake at all time points. Brain uptake is the ratio of brain levels (as moles/g tissue) over plasma levels (as moles/up and is expressed as ul/g. One gram of brain tissue contains approximately 20 ul of plasma. Thus, any brain uptake value above 20 ul/g is indicative of delivery across the blood-brain barrier to the brain parenchyma. For example, the brain uptake values for EUK-418 are in excess of about 100 ul/g.

### Example 7

### Catalytic Activities of Compounds

### Methods

The compounds designated EUK-418, EUK-423, EUK-425, EUK-450, EUK-451, EUK-452 and EUK-453 were tested for catalase activity against a known standard catalase mimetic, which is a structurally-unrelated salen-metal compound designated EUK-189. Catalase, superoxide dismutase (SOD), and peroxidase activities were determined essentially as described by Doctrow et al., J. Med. Chem. 45, 4549-4558, 2002, which is hereby incorporated by reference.

More particularly, catalase assay was performed by incubating compounds with hydrogen peroxide for 20 minutes, and measuring remaining hydrogen peroxide levels colorimetrically in peroxidase-coupled reactions. Each compound was present in the reactions at a final concentration of 10 μM, diluted from stock solutions in DMSO, with the exception of the positive control EUK-189 compound which was diluted from a stock solution in H₂O.

### Results

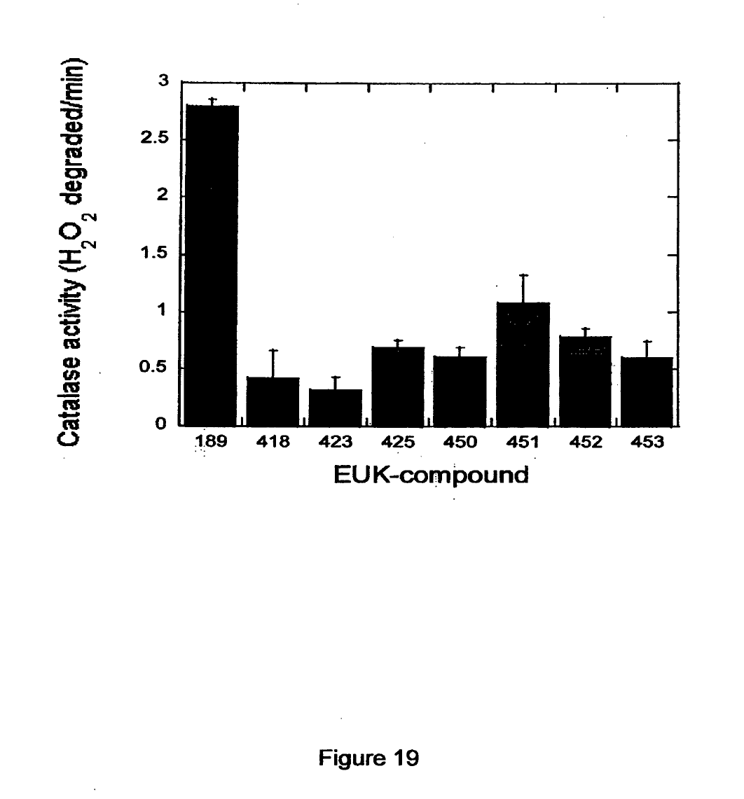

All of the compounds tested exhibited significant catalase activities (FIG. 19), albeit less than that of the reference compound EUK-189. Additionally, the tested compounds had significant SOD activity (Table 3) and peroxidase activity (not shown), albeit less than that of EUK-189.

TABLE 3
Superoxide dismutase activities of compounds

| Compound Designation | Cuvette IC₅₀ (μM) | Microplate IC₅₀ (μM) |
|---|---|---|
| EUK-189 | 0.550 | 00.824 |
| EUK-207 | 0.150 | 0.0152 |
| EUK-418 | 8.280 | 0.382 |
| EUK-423 | 17.29 | 4.100 |
| EUK-425 | 32.50 | 0.439 |
| EUK-450 | 9.980 | 3.120 |
| EUK-451 | 29.06 | 2.390 |
| EUK-452 | 20.05 | 9.050 |
| EUK-453 | 60.88 | 2.300 |

### Example 8

### Efficacy of Compounds in a Model of Parkinson's Disease In Vivo

The in vivo effects of these compounds in the MPTP model for Parkinson's disease and the pharmacokinetic properties of the compounds in this model are determined following both intravenous and oral administration of the compounds to mice. Genotoxicity (e.g., using the Ames test; mouse lymphoma assay) and chronic oral toxicity (e.g., by determining blood chemistry, weight gain, and histopathology after a two months treatment with a compound) are also determined to ensure safety of the compounds when administered to animals. Such studies further validate the porphyrin compounds of the present invention as therapeutic compounds for the treatment of neurodegenerative diseases, especially Parkinson's Disease.

The MPTP model of neurotoxicity in mice is probably the most widely used animal model for Parkinson's disease. It offers a number of advantages for studying not only potential mechanisms of neurodegeneration, but also for identifying potential therapeutic approaches for the human disease (Grunblatt et al., J. Neural. 247 Suppl 2, 95-102, 2000; Teismann and Ferger, Synapse 39(2), 167-174, 2001; Kaur et al., Neuron 37(6), 899-909, 2003; all references incorporated herein by reference). The various manifestations of pathology appear relatively rapidly (3-7 days), they are relatively well reproducible, and the small size of the animals allows the use of small amounts of drugs.

### 1. Animal Treatments

Adult male mice (90 day old, C57BL/6J, Jackson Laboratories, Ithaca, N.Y.) are housed under standard laboratory conditions with a 12-h light/dark cycle and free access to food and water.

### a) Administration of Injectable Formulations

In one example, the ability of EUK-418 formulated for injection to provide neuroprotection against MPTP toxicity is determined. Sub-acute MPTP treatment with EUK-418 comprises five, six, seven or eight daily i.p. injections of EUK-418 formulated for injection at concentrations of about 0.3 mg/kg body weight, 3.0 mg/kg and 30 mg/kg to different groups of animals (10-12 mice per group) 1 hr before MPTP (25 mg/kg in saline, s.c.). Mice receive subcutaneous injections of MPTP (Sigma Chemical, St. Louis, Mo., 25 mg/kg in saline) daily for 5 consecutive days.

In another example, the ability of EUK-423 to provide neuroprotection against MPTP toxicity is determined. Sub-acute MPTP treatment with EUK-423 comprises five, six, seven or eight daily i.p. injections of EUK-423 formulated for injection at concentrations of about 0.3 mg/kg body weight, 3.0 mg/kg and 30 mg/kg to different groups of animals (10-12 mice per group) 1 hr before MPTP (25 mg/kg in saline, s.c.) administered daily for the first five days.

In another example, the ability of EUK-424 to provide neuroprotection against MPTP toxicity is determined. Sub-acute MPTP treatment with EUK-424 comprises five, six, seven or eight daily i.p. injections of EUK-424 formulated for injection at concentrations of about 0.3 mg/kg body weight, 3.0 mg/kg and 30 mg/kg to different groups of animals (10-12 mice per group) 1 hr before MPTP (25 mg/kg in saline, s.c.) administered daily for the first five days.

In another example, the ability of EUK-425 to provide neuroprotection against MPTP toxicity is determined. Sub-acute MPTP treatment with EUK-425 comprises five, six, seven or eight daily i.p. injections of EUK-425 formulated for injection at concentrations of about 0.3 mg/kg body weight, 3.0 mg/kg and 30 mg/kg to different groups of animals (10-12 mice per group) 1 hr before MPTP (25 mg/kg in saline, s.c.), administered daily for the first five days.

In another example, the ability of EUK-426 to provide neuroprotection against MPTP toxicity is determined. Sub-acute MPTP treatment with EUK-426 comprises five, six, seven or eight daily i.p. injections of EUK-426 formulated for injection at concentrations of about 0.3 mg/kg body weight, 3.0 mg/kg and 30 mg/kg to different groups of animals (10-12 mice per group) 1 hr before MPTP (25 mg/kg in saline, s.c.), administered daily for the first five days.

In another example, the ability of EUK-450 to provide neuroprotection against MPTP toxicity is determined. Sub-acute MPTP treatment with EUK-450 comprises five, six, seven or eight daily i.p. injections of EUK-450 formulated for injection at concentrations of about 0.3 mg/kg body weight, 3.0 mg/kg and 30 mg/kg to different groups of animals (10-12 mice per group) 1 hr before MPTP (25 mg/kg in saline, s.c.), administered daily for the first five days.

In another example, the ability of EUK-451 to provide neuroprotection against MPTP toxicity is determined. Sub-acute MPTP treatment with EUK-451 comprises five, six, seven or eight daily i.p. injections of EUK-451 formulated for injection at concentrations of about 0.3 mg/kg body weight, 3.0 mg/kg and 30 mg/kg to different groups of animals (10-12 mice per group) 1 hr before MPTP (25 mg/kg in saline, s.c.), administered daily for the first five days.

In another example, the ability of EUK-452 to provide neuroprotection against MPTP toxicity is determined. Sub-acute MPTP treatment with EUK-452 comprises five, six, seven or eight daily i.p. injections of EUK-452 formulated for injection at concentrations of about 0.3 mg/kg body weight, 3.0 mg/kg and 30 mg/kg to different groups of animals (10-12 mice per group) 1 hr before MPTP (25 mg/kg in saline, s.c.), administered daily for the first five days.

In another example, the ability of EUK-453 to provide neuroprotection against MPTP toxicity is determined. Sub-acute MPTP treatment with EUK-453 comprises five, six, seven or eight daily i.p. injections of EUK-453 formulated for injection at concentrations of about 0.3 mg/kg body weight, 3.0 mg/kg and 30 mg/kg to different groups of animals (10-12 mice per group) 1 hr before MPTP (25 mg/kg in saline, s.c.), administered daily for the first five days.

### b) Administration of Oral Formulations

In a further example, the ability of an orally-administered compound to provide neuroprotection against MPTP toxicity is determined. Compounds are administered orally by gavage to determine a dose-response curve, and to determine the lowest effective daily dose of the compound(s).

In one example, the ability of EUK-418 to provide neuroprotection against MPTP toxicity is determined. Sub-acute MPTP treatment with EUK-418 comprises five, six, seven, eight, nine or ten daily doses by intragastric gavage of EUK-418 at concentrations of about 4.0 mg/kg, 40 mg/kg and 100 mg/kg to different groups of animals (10-12 mice per group) 1 hr before MPTP (25 mg/kg in saline, s.c.), administered daily for the first five days.

In another example, the ability of EUK-423 to provide neuroprotection against MPTP toxicity is determined. Sub-acute MPTP treatment with EUK-423 comprises five, six, seven, eight, nine or ten daily doses by intragastric gavage of EUK-423 at concentrations of about 4.0 mg/kg, 40 mg/kg and 100 mg/kg to different groups of animals (10-12 mice per group) 1 hr before MPTP (25 mg/kg in saline, s.c.), administered daily for the first five days.

In another example, the ability of EUK-424 to provide neuroprotection against MPTP toxicity is determined. Sub-acute MPTP treatment with EUK-424 comprises five, six, seven, eight, nine or ten daily doses by intragastric gavage of EUK-424 at concentrations of about 4.0 mg/kg, 40 mg/kg and 100 mg/kg to different groups of animals (10-12 mice per group) 1 hr before MPTP (25 mg/kg in saline, s.c.), administered daily for the first five days.

In another example, the ability of EUK-425 to provide neuroprotection against MPTP toxicity is determined. Sub-acute MPTP treatment with EUK-425 comprises five, six, seven, eight, nine or ten daily doses by intragastric gavage of EUK-425 at concentrations of about 4.0 mg/kg, 40 mg/kg and 100 mg/kg to different groups of animals (10-12 mice per group) 1 hr before MPTP (25 mg/kg in saline, s.c.), administered daily for the first five days.

In another example, the ability of EUK-426 to provide neuroprotection against MPTP toxicity is determined. Sub-acute MPTP treatment with EUK-426 comprises five, six, seven, eight, nine or ten daily doses by intragastric gavage of EUK-426 at concentrations of about 4.0 mg/kg, 40 mg/kg and 100 mg/kg to different groups of animals (10-12 mice per group) 1 hr before MPTP (25 mg/kg in saline, s.c.) administered daily for the first five days.

In another example, the ability of EUK-450 to provide neuroprotection against MPTP toxicity is determined. Sub-acute MPTP treatment with EUK-450 comprises five, six, seven eight, nine or ten daily doses by intragastric gavage of EUK-450 at concentrations of about 4.0 mg/kg, 40 mg/kg and 100 mg/kg to different groups of animals (10-12 mice per group) 1 hr before MPTP (25 mg/kg in saline, s.c.), administered daily for the first five days.

In another example, the ability of EUK-451 to provide neuroprotection against MPTP toxicity is determined. Sub-acute MPTP treatment with EUK-451 comprises five, six, seven, eight, nine or ten daily doses by intragastric gavage of EUK-451 at concentrations of about 4.0 mg/kg, 40 mg/kg and 100 mg/kg to different groups of animals (10-12 mice per group) 1 hr before MPTP (25 mg/kg in saline, s.c.), administered daily for the first five days.

In another example, the ability of EUK-452 to provide neuroprotection against MPTP toxicity is determined. Sub-acute MPTP treatment with EUK-452 comprises five, six, seven, eight, nine or ten daily doses by intragastric gavage of EUK-452 at concentrations of about 4.0 mg/kg, 40 mg/kg and 100 mg/kg to different groups of animals (10-12 mice per group) 1 hr before MPTP (25 mg/kg in saline, s.c.).

In another example, the ability of EUK-453 to provide neuroprotection against MPTP toxicity is determined. Sub-acute MPTP treatment with EUK-453 comprises five, six, seven eight, nine or ten daily doses by intragastric gavage of EUK-453 at concentrations of about 4.0 mg/kg, 40 mg/kg and 100 mg/kg to different groups of animals (10-12 mice per group) 1 hr before MPTP (25 mg/kg in saline, s.c.), administered daily for the first five days.

### 2. Assay Readouts

(i) [³H]Mazindol Binding in Solution

To assess the effects of a compound of the present invention on MPTP-induced toxicity against dopaminergic neurons, [³H]-mazindol binding is determined. Mazindol is a dopamine transporter antagonist commonly used as a marker of dopaminergic neuron integrity (Sundstrom et al., Brain Res Bull 21(2): 257-263, 1988; Donnan et al., Brain Res 504(1), 64-71, 1989; both citations being incorporated by reference). Striatal mazindol binding is known as a marker of dopaminergic terminals (Javitch et al., Eur. J. Pharmacol. 90, 461-462, 1983; Sundstrom et al., Brain Res Bull 21(2): 257-263, 1988; both citations being incorporated by reference) because their destruction results in a decrease in dopamine transporters, and a concomitant decrease in [³H]-mazindol binding to the pore.

Animals are sacrificed following administration of the final dose of compound, their striata are homogenized in ice-cold buffer (50 mM Tris/HCl, pH 7.4), and a membrane rich fraction is obtained by centrifuging the homogenate at 15,000 rpm for 20 min. Pellets are resuspended in binding buffer (50 mM Tris/HCl, 300 mM NaCl and 5 mM KCl, pH 7.4), and incubated with [³H]-mazindol (NEN, Boston, Mass., 17 Ci/mol) for 2-h at 4° C.

To determine non-specific binding, another dopamine transporter ligand, nomifensine (Research Biochemical Int., Natick, Mass., 2.8 μM) is added.

Incubation is terminated by filtration through glass fiber filters (Whatman GF/C; Whatman International Ltd., Maidstone, England) using a Brandel cell harvester (Biochemical Research and Development Laboratories Inc., Gaithersberg, Md.). Radioactivity bound to the filters is determined with a liquid scintillation counter (Beckman Instruments Inc., Fullerton, Calif.).

Protein concentration is determined for each sample using the Bradford method, in order to correct for differences in the amount of tissue dissected.

Efficacy of a compound of the invention is demonstrated by reduced specific binding of [³H]-mazindol relative to the binding observed for samples taken from control animals that received MPTP but no metalloporphyrin derivative.

(ii) [³H]-Mazindol Binding Autoradiography

Quantitative autoradiography of [³H]-mazindol binding is performed on frozen-thawed brain sections essentially as described by Puschban et al., Neuroscience 95, 377-388, 2000, which is incorporated herein by reference. Immediately prior to their being sacrificed at the end of treatment with compound, rats are anaesthetized with pentobarbital and perfused transcardially with 300 ml ice-cold 5% dextrose-saline. Brains are removed and snap-frozen in isopentane. Coronal sections (20 μm) are cut at −20° C. and mounted onto gelatine-coated glass slides. Alternate sections are allocated to slides for total or non-specific binding. Sections are dried in a stream of cold air.

Adjacent sections for total and non-specific binding are incubated with [³H]mazindol. Briefly, sections for [³H]mazindol binding are thawed at room temperature and binding is performed at 4° C. in an assay buffer consisting of 50 mM Tris/HCl, 300 mM NaCl and 5 mM KCl (pH 7.9). Sections for total binding are incubated for 45 min in buffer containing 4 nM [³H]mazindol and 0.3 mM desmethylimipramine to prevent binding to noradrenaline uptake sites. Non-specific binding is assessed by incorporating 10 μM mazindol. Incubation is terminated by two consecutive washes in Tris buffer (1 min each). Slides and tritium standards (Amersham) are exposed to tritium-sensitive film at 4° C. for about six weeks.

Efficacy of a compound of the invention is demonstrated by reduced specific binding of [³H]-mazindol relative to the binding observed for samples taken from control animals that received MPTP but no metalloporphyrin derivative.

### Example 9

### Assay for Protection Against Paraquat-Mediated Dopaminergic Neuron Death in the Substantia Nigra

### 1. Materials

3-(4,5-Dimethylthiazol-2-yl)-2,5-diphenyltetrazolium bromide (MTT),1,1′-dimethyl-4,4′-bipyridium dichloride (paraquat), protease inhibitor mixture, lactacystin, and monoclonal anti-β-actin antibody are purchased from Sigma. Polyvinylidene difluoride membrane and SDS-PAGE gels are obtained from Bio-Rad. Rabbit anti-phospho-stress-activated protein kinase/JNK (Thr¹⁸³/Tyr¹⁸⁶), anti-phospho-c-Jun (Ser⁶³), anti-cleaved caspase-3, and anti-caspase-3 antibodies are purchased from Cell Signaling Technology, Beverly, Mass. Rabbit and sheep anti-tyrosine hydroxylase polyclonal antibodies are obtained from Chemicon, Temecula, Calif. Media and sera are purchased from Invitrogen. Osmotic minipumps (Alzet 2004) are from Alza Scientific Products, Mountain View, Calif.

### 2. Methods

a) Cell Culture

The rat dopaminergic cell line 1RB₃AN₂₇ (N27) is grown in RPMI 1640 medium supplemented with 10% fetal calf serum (Invitrogen), 100 units/ml penicillin, and 100 μg/ml streptomycin.

Cell viability is determined by MTT incorporation essentially as described by Peng et al., J. Biol. Chem. 277, 44285-44291, 2002, the contents of which are incorporated herein by reference.

DNA fragmentation is examined by terminal deoxynucleotidyl transferase-mediated dUTP nick end-labeling (TUNEL) analysis with an in situ cell death detection kit (Roche Molecular Biochemicals) according to the manufacturer's instructions (see also Peng et al., J. Biol. Chem. 277, 44285-44291, 2002). Stained cells are counted in 10 randomly chosen microscopic fields (at least 500 cells). Data are expressed as the mean±S.E. of the percentage of total cells that display TUNEL staining.

To evaluate the effect of the metalloporphyrin derivatives of the invention on cell death, the compounds are added 1 hr prior to paraquat or lactacystin.

Caspase-3 activity is performed using a commercially available kit from Bio-Rad, Hercules, Calif. as described by Peng et al., J. Biol. Chem. 277, 44285-44291, 2002. Briefly, cells are pelleted and subsequently lysed. Whole supernatant following sedimentation is incubated with the synthetic substrate cabobenzoxy-Asp-Glu-val-Asp-7-amino-4-trifluoromethylcoumarin for 2 h at 37° C. Measurements are made on a fluorescent microplate reader using filters for excitation (400 nm) and detection of emitted light (530 nm). Serial dilutions of amino-4-trifluoromethylcoumarin are used as standards. A negative control in which caspase-3 inhibitor (Ac-DEVD-chloromethyl ketone) is added and a positive control containing apopain are used to test the efficacy of the assay.

b) Primary Mesencephalic Cultures

Primary mesencephalic cell cultures are prepared from embryonic gestation day 14-15 mouse embryos as described by Peng et al., J. Biol. Chem. 279, 32626-32632, 2004, the contents of which are incorporated by reference. Briefly, dissociated cells are seeded at 7×10⁵ cells per well onto poly-D-lysine-coated 24-well culture plates. Cultures are maintained at 37° C. in a humidified atmosphere containing 95% air and 5% carbon dioxide, in Neurobasal medium (Invitrogen) containing 2% B27 supplement, 2 mM glutamate, 100 units/ml penicillin, and 100 μg/ml streptomycin. After 4 days, one-half of the medium is replaced with fresh medium. Cells are grown an additional 2 days and then treated with 40 μM paraquat for 18 or 24 h. The number of tyrosine hydroxylase (TH)-positive neurons in mesencephalic cultures is determined as described by Peng et al., J. Biol. Chem. 279, 32626-32632, 2004. The specificity of neurotoxicity is analyzed by double label immunostaining with anti-TH antibody and antibodies against phospho-JNK, phospho-c-Jun, and cleaved caspase-3, respectively, as described by Peng et al., J. Biol. Chem. 279, 32626-32632, 2004. Experiments are repeated with cultures isolated from at least about four independent dissections.

c) Immunocytochemistry

Cultures are fixed with paraformaldehyde in phosphate-buffered saline and permeabilized with 0.3% Triton X-100 in phosphate-buffered saline as described previously by Peng et al., J. Biol. Chem. 279, 32626-32632, 2004. Primary antibodies included the following: sheep polyclonal anti-TH (1:500), rabbit polyclonal anti-phospho-JNK (1:100), rabbit polyclonal anti-phospho-c-Jun (1:100), and rabbit polyclonal anti-cleaved caspase-3 (1:200).

Secondary antibodies are rhodamine-conjugated rat-absorbed donkey anti-rabbit IgG (Jackson ImmunoResearch; 1:200) and fluorescein isothiocyanate-conjugated goat anti-sheep IgG (Vector, Burlingame, Calif., (1:200).

4′,6-Diamidino-2-phenylindole (DAPI) (Vector) is used to counterstain nuclei.

Control experiments include omitting primary antibody.

d) Administration of Compounds

Eight-week-old male C57BL/6 mice (Jackson Laboratory, Bar Harbor, Me.) are anesthetized with 4% isoflurane in 70% N₂O/30% O₂ and subcutaneously implanted with an osmotic minipump containing either 5% mannitol (as vehicle control) or 15 mM EUK-189 (dissolved in 5% mannitol). Pumps deliver the compounds at a rate of 0.25 μl/h for a 28-day period. The calculated compound infusion rate is about 0.09 μmol/day.

e) Paraquat Administration

Mice are intraperitoneally injected with either saline or 7 mg/kg paraquat (dissolved in saline) at 2-day intervals for a total of 10 doses. Animals are killed at day 7 or 8 after the last administration as described by Peng et al., J. Biol. Chem. 279, 32626-32632, 2004. Experimental protocols are in accordance with the National Institutes of Health Guidelines for Use of Live Animals and are approved by the Animal Care and Use Committee at the Buck Institute of Age Research.

f) Stereological SN TH-Positive Neuron Counts

Littermates are fixed by perfusion as described by Peng et al., J. Biol. Chem. 279, 32626-32632, 2004. Cryostat-cut sections (40 μm) are taken through the entire midbrain. TH-positive neurons are immunolabeled by incubating the tissue sections successively with a rabbit polyclonal anti-TH antibody (1:200) and biotinylated horse anti-rabbit IgG (1:200, Vector Laboratories) and following the staining procedure outlined by the manufacturers of Vectastain ABC kit (Vector Laboratories) in combination with 3,3′-diaminobenzidine (DAB) reagents. The total number of TH-positive neurons in the substantia nigra pars compacta is determined from four to five littermates per group by using the optical fractionator method, an unbiased stereological technique of cell counting as described by Peng et al., J. Biol. Chem. 279, 32626-32632, 2004.

g) Western Blot Analysis

Total protein is isolated from brain tissue as described by Peng et al., J. Biol. Chem. 279, 32626-32632, 2004. Protein concentration of the supernatant is determined using a commercially available protein assay kit (Bio-Rad). Equal concentrations of protein extracts are electrophoretically resolved on SDS-polyacrylamide gels and transferred to polyvinylidene difluoride membranes. Primary antibodies for Western blot analysis are used at the following dilutions: phospho-JNK (1:1000), phospho-c-Jun (1:1000), caspase-3 (1:1000), and β-actin (1:5000). Detection is performed using horseradish peroxidase-conjugated secondary antibody and an ECL kit (Amersham Biosciences).

h) Statistical Analysis

Data are expressed as mean±S.E. for the number (n) of independent experiments performed. Differences among the means for all experiments described are analyzed using one- or two-way analysis of variance. Newman-Keuls post-hoc analysis is employed when differences were observed by analysis of variance testing (p<0.05).

### 3. Expected Assay Results

N27 is an immortalized dopaminergic neuronal cell line isolated from fetal rat mesencephalic cultures that produces dopamine and expresses the dopamine-synthesizing enzyme tyrosine hydroxylase (TH) and the dopamine transporter (DAT). This cell line is an accepted model to study the potential role of paraquat on the JNK signaling pathway, because it relates to dopaminergic cell death associated with Parkinson's Disease (PD). Treatment of N27 cells with 400 μM paraquat for 18-24 h increases caspase-3 activation, cell death, and DNA fragmentation compared with untreated controls. However, in the presence of an amount of a metalloporphyrin of the present invention 1 hr before addition of paraquat, caspase-3 activation, cell death, and DNA fragmentation are significantly inhibited if the compound is protective against dopaminergic neuron death.

Lactacystin is a selective proteasome inhibitor that does not significantly inhibit other proteases, even at high concentration. To study whether the effects of the metalloporphyrin derivatives of the present invention are specific for oxidative stress-induced cell death, N27 cells are treated with the compounds for 1 hr prior to treatment with 5 μM lactacystin. Cell death and DNA fragmentation are analyzed by MTT and TUNEL staining methods at 24 hr post-treatment.

Paraquat-generated superoxide leads to activation of the JNK signaling pathway resulting in subsequent dopaminergic neuronal apoptosis. To assess the neuroprotective ability of the metalloporphyrin derivatives of the present invention in relation to paraquat-induced cell death on a cellular level in primary dopamine midbrain neurons, the effects of the compounds on paraquat-treated primary mesencephalic cultures are examined via dual immunofluorescence with antibodies specific for TH and either phospho-JNK, phospho-c-Jun, or cleaved caspase-3, respectively, coupled with 4′,6-diamidino-2-phenylindole staining. Cultures are pretreated with compounds (0.5 μM, 1 μM, 2 μM and 3 μM) 1 hr prior to treatment with 40 μM paraquat. A reduction in co-localization of phospho-JNK, phospho-c-Jun, and activated caspase-3 with TH-positive neurons after 18 h of paraquat treatment is indicative of a protective effect.

Cells are also stained for TH at 24 h following paraquat treatment, and TH-positive neurons are counted. Compounds that protect TH-positive neurons from paraquat-induced cell death are desired.

To examine whether a compound of the present invention attenuates the selective loss of nigrostriatal dopamine neurons after paraquat administration in vivo, mice are implanted with pumps containing either 5% mannitol (as vehicle control) or the metalloporphyrin derivative 1 day prior to paraquat treatment. Exposure of mice to paraquat alone should produce a substantial loss of nigral dopamine neurons when compared with unlesioned controls, whereas subcutaneous administration of a metalloporphyrin compound of the invention should significantly attenuate the loss of nigral dopamine neurons when examined at day 8 following the last paraquat treatment.

To investigate whether inhibition of the JNK apoptotic cascade contributes to the neuroprotection conferred by a metalloporphyrin derivative of the present invention following paraquat injection, the levels of phospho-JNK, phospho-c-Jun, and cleaved caspase-3 are detected by Western blot analysis of substantia nigra. The levels of phosphorylated JNK, phosphorylated c-Jun, and cleaved caspase-3 should be enhanced in this tissue prepared from paraquat-treated mice compared with the same tissue prepared from mice in the saline treatment group. However, pretreatment with a metalloporphyrin compound of the invention should partially or completely suppress paraquat-induced increases in phosphorylation of JNK and c-Jun and caspase-3 cleavage.

### Example 10

### Toxicology

Metalloporphyrins are known to be able to cleave DNA when they are positively charged. However, neutral metalloporphyrins are non-genotoxic (U.S. Pat. No. 6,403,788). Preferred means for determining the potential genotoxicity of a compound of the present invention are the Ames test and the mouse lymphoma assay.

### Determination of Oral Toxicity

Preferred means for evaluating the safety of chronic oral administration of a compound of the present invention are by monitoring blood chemistry, weight gain, and histopathology after a two months treatment of animals with daily dosages of a compound being tested. During such trials, the weights of mice are monitored 5 times per week, and weekly averages for treated versus untreated mice are recorded. Body weight is a non-invasive, highly predictive way of assessing chronic toxicity. After two months, mice are sacrificed, their blood is collected for determining blood chemistry including residual levels of the administered compounds. Major body organs including brain (target organ), liver, kidney and heart are also collected and frozen for subsequent histopathological evaluation.

We claim:

1. A composition for inhibiting, delaying or preventing apoptosis comprising a low molecular weight porphyrin derivative that inhibits, prevents or delays binding of a ligand of a mitochondrial benzodiazepine receptor, wherein said low molecular weight porphyrin derivative has a structure represented by Structural Formula I: 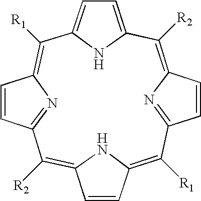
wherein one or both occurrences of R1 is aliphatic or aromatic and wherein one or both occurrences of R2 is hydrogen or aliphatic.

2. The composition according to claim 1 wherein one or both R1 is selected from the group consisting of lower alkyl, aryl and mixtures thereof.

3. The composition according to claim 1 wherein one or both R2 is selected from the group consisting of hydrogen, lower alkyl and mixtures thereof.

4. The composition according to claim 1 wherein one or both R1 is selected from the group consisting of lower alkyl, aryl and mixtures thereof and wherein one or both R2 is selected from the group consisting of hydrogen, lower alkyl and mixtures thereof.

5. The composition according to claim 1 wherein R1 is selected from aryl, n-alkyl, branched alkyl, cycloalkyl consisting of one to six carbons and mixtures thereof, and wherein R2 is selected from hydrogen, n-alkyl, branched alkyl consisting of one, two or three carbons and mixtures thereof.

6. The composition according to claim 1 wherein R1 and/or R2 are selected independently from the group consisting of hydrogen, methyl, ethyl, n-propyl, iso-propyl, cyclopropyl, 4-tetrahydropyrano, cyclohexyl, phenyl and 3,4-methoxyphenyl.

7. The composition according to claim 1 wherein R1 and/or R2 are selected independently from the group consisting of n-propyl, 4-tetrahydropyrano, cyclohexyl and 3,4-methoxyphenyl.

8. The composition according to claim 1 wherein the low molecular weight porphyrin derivative is complexed with a first row transition metal.

9. The composition according to claim 1 wherein the transition metal is selected from the group consisting of manganese, iron, cobalt, copper, nickel and zinc.

10. The composition according to claim 8 wherein the transition metal is manganese.

11. The composition according to claim 1 complexed with an axial ligand consisting of a monovalent anion.

12. The composition according to claim 11 wherein the monovalent anion is a halogen or organic anion.

13. The composition according to claim 12 wherein the monovalent anion is chloride.

14. The composition according to claim 12 wherein the organic anion is selected from the group consisting of acetate, propionate, butyrate, formate and triflate.

15. The composition according to claim 14 wherein the organic anion is acetate.

16. A composition for inhibiting, delaying or preventing apoptosis comprising a low molecular weight porphyrin derivative that inhibits, prevents or delays binding of a ligand of a mitochondrial benzodiazepine receptor, wherein said low molecular weight porphyrin derivative has a structure represented by Structural Formula II: 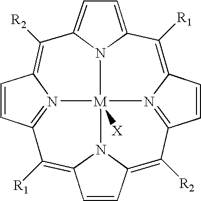
wherein:
a) each R1 is the same and selected from the group consisting of methyl, ethyl, n-propyl, iso-propyl, cyclopropyl, 4-tetrahydropyrano, cyclohexyl, phenyl and 3,4-methoxyphenyl;
b) each R2 is the same and selected from hydrogen, methyl, ethyl and iso-propyl;
c) M is a transition metal selected from the group consisting of manganese, chromium, iron, cobalt, copper, titanium, vanadium, rubidium, osmium, nickel and zinc; and
d) X is an axial ligand consisting of halogen or organic anion.

17. The composition according to claim 16 wherein M is manganese and X is chloride.

18. The composition according to claim 16 wherein M is manganese and X is acetate.

19. The composition according to claim 16 wherein R1 is selected from the group consisting of n-propyl, 4-tetrahydropyrano, cyclohexyl and 3,4-methoxyphenyl.

20. A composition for inhibiting, delaying or preventing apoptosis comprising a low molecular weight porphyrin derivative that inhibits, prevents or delays binding of a ligand of a mitochondrial benzodiazepine receptor, wherein said low molecular weight porphyrin derivative has a structure represented by Structural Formula III: 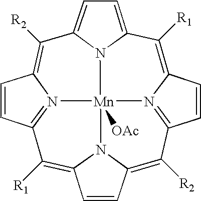
wherein:
a) each R1 is the same and selected from the group consisting of methyl, ethyl, iso-propyl, cyclopropyl, cyclohexyl, phenyl and 3,4-methoxyphenyl; and
b) each R2 is the same and selected from hydrogen, methyl, ethyl and iso-propyl.

21. The composition according to claim 20 wherein R1 is selected from the group consisting of n-propyl, 4-tetrahydropyrano, cyclohexyl and 3,4-methoxyphenyl.

22. A composition for inhibiting, delaying or preventing apoptosis comprising a low molecular weight porphyrin derivative that inhibits, prevents or delays binding of a ligand of a mitochondrial benzodiazepine receptor, wherein said low molecular weight porphyrin derivative has a structure represented by Structural Formula IV: 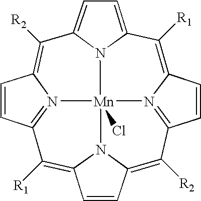
wherein:
a) each R1 is the same and selected from the group consisting of n-propyl, 4-tetrahydropyrano and cyclohexyl; and
b) each R2 is hydrogen.

23. A composition for inhibiting, delaying or preventing apoptosis low molecular weight porphyrin derivative that that inhibits, prevents or delays binding of a ligand of a mitochondrial benzodiazepine receptor, wherein the porphyrin derivative is selected from the group consisting of:
a) {[{(Porphine-5,15-diyl)bis[cyclopropyl-diyl]}](2-)-N²¹,N²²,N²³,N²⁴} manganese(III) acetate (EUK-418);
b) {[{(Porphine-5,15-diyl)bis[benzyl-diyl]}](2-)-N²¹,N²²,N²³,N²⁴} manganese(III) acetate (EUK-423);
c) (5,10,15,20-Tetraisopropylporphyrinato)manganese (III) acetate (EUK-424);
d) (5,10,15,20-Tetraethylporphyrinato)manganese (III) acetate (EUK-425);
e) (5,10,15,20-Tetramethylporphyrinato)manganese (III) acetate (EUK-426);
f) {[{(Porphine-5,15-diyl)bis[benzene-1,4 diyl (4-methyl-oxy)]}](2-)-N²¹, N²²,N²³,N²⁴} manganese(III) acetate (EUK-450);
g) {[{(Porphine-5,15-diyl)bis[4-Tetrahydropyrano-diyl]}](2-)-N²¹,N²²,N²³,N²⁴} manganese(III) chloride (EUK-451);
h) {[{(Porphine-5,15-diyl)bis[cyclohexyl-diyl]}](2-)-N²¹,N²²,N²³,N²⁴} manganese(III) chloride (EUK-452); and
i) {[{(Porphine-5,15-diyl)bis[propyl-diyl]}](2-)-N²¹,N²²,N²³,N²⁴} manganese(III) chloride (EUK-453).

24. The composition according to claim 23 wherein the low molecular weight porphyrin derivative that inhibits, prevents or delays binding of a ligand of a mitochondrial benzodiazepine receptor is {[{(Porphine-5,15-diyl)bis[cyclopropyl-diyl]}](2-)-N²¹,N²²,N²³,N²⁴} manganese(III) acetate (EUK-418).

25. The composition according to claim 23 wherein the low molecular weight porphyrin derivative that inhibits, prevents or delays binding of a ligand of a mitochondrial benzodiazepine receptor is {[{(Porphine-5,15-diyl)bis[benzyl-diyl]}](2-)-N²¹,N²²,N²³,N²⁴} manganese(III) acetate (EUK-423).

26. The composition according to claim 23 wherein the low molecular weight porphyrin derivative that inhibits, prevents or delays binding of a ligand of a mitochondrial benzodiazepine receptor is (5,10,15,20-Tetraisopropylporphyrinato)manganese (III) acetate (EUK-424).

27. The composition according to claim 23 wherein the low molecular weight porphyrin derivative that inhibits, prevents or delays binding of a ligand of a mitochondrial benzodiazepine receptor is (5,10,15,20-Tetraethylporphyrinato)manganese (III) acetate (EUK-425).

28. The composition according to claim 23 wherein the low molecular weight porphyrin derivative that inhibits, prevents or delays binding of a ligand of a mitochondrial benzodiazepine receptor is (5,10,15,20-Tetramethylporphyrinato)manganese (III) acetate (EUK-426).

29. The composition according to claim 23 wherein the low molecular weight porphyrin derivative that inhibits, prevents or delays binding of a ligand of a mitochondrial benzodiazepine receptor is {[{(Porphine-5,15-diyl)bis[benzene-1,4 diyl (4-methyl-oxy)]}](2-)-N²¹,N²²,N²³,N²⁴} manganese(III) acetate (EUK-450).

30. The composition according to claim 23 wherein the low molecular weight porphyrin derivative that inhibits, prevents or delays binding of a ligand of a mitochondrial benzodiazepine receptor is {[{(Porphine-5,15-diyl)bis[4-Tetrahydropyrano-diyl]}](2-)-N²¹,N²²,N²³,N²⁴} manganese(III) chloride (EUK-451).

31. The composition according to claim 23 wherein the low molecular weight porphyrin derivative that inhibits, prevents or delays binding of a ligand of a mitochondrial benzodiazepine receptor is {[{(Porphine-5,15-diyl)bis[cyclohexyl-diyl]}](2-)-N²¹,N²²,N²³,N²⁴} manganese(III) chloride (EUK-452).

32. The composition according to claim 23 wherein the low molecular weight porphyrin derivative that inhibits, prevents or delays binding of a ligand of a mitochondrial benzodiazepine receptor is {[{(Porphine-5,15-diyl)bis[propyl-diyl]}] (2-)-N²¹,N²²,N²³,N²⁴} manganese(III) chloride (EUK-453).

33. A composition comprising {[{(Porphine-5,15-diyl)bis[benzene-1,4 diyl (4-methyl-oxy)]}](2-)-N²¹,N²²,N²³,N²⁴} manganese(III) acetate (EUK-450).

34. A composition comprising {[{(Porphine-5,15-diyl)bis[4-Tetrahydropyrano-diyl]}](2-)-N²¹,N²²,N²³,N²⁴} manganese(III) chloride (EUK-451).

35. A composition comprising {[{(Porphine-5,15-diyl)bis[cyclohexyl-diyl]}](2-)-N²¹,N²²,N²³,N²⁴} manganese(III) chloride (EUK-452).

36. A composition comprising {[{(Porphine-5,15-diyl)bis[propyl-diyl]}](2-)-N²¹,N²²,N²³,N²⁴} manganese(III) chloride (EUK-453).

37. A pharmaceutical formulation comprising one or more pharmaceutically acceptable carriers, diluents or excipients and a therapeutically effective amount of at least one low molecular weight porphyrin derivative according to claim 16.

38. The pharmaceutical formulation of claim 37 wherein the therapeutically effective amount of at least one low molecular weight porphyrin derivative compound is sufficient to inhibit, prevent or reduce opening of a mitochondrial permeability transition pore (mPTP) in a cell.

39. The pharmaceutical formulation of claim 37 wherein the therapeutically effective amount of at least one low molecular weight porphyrin derivative compound is sufficient to inhibit, prevent or reduce mitochondrial membrane depolarization in a cell.

40. The pharmaceutical formulation of claim 37 wherein the therapeutically effective amount of at least one low molecular weight porphyrin derivative compound is sufficient to inhibit, prevent or reduce the release of calcium and/or cytochrome C from a cell.

41. The pharmaceutical formulation of claim 37 wherein the therapeutically effective amount of at least one low molecular weight porphyrin derivative compound is sufficient to reduce, delay or inhibit apoptosis of cells.

42. A method of treating a disease associated with apoptosis in a mammal said method comprising administering to the mammal an amount of a pharmaceutical formulation according to claim 37 effective to inhibit, delay or prevent apoptosis.

43. The method according to claim 42 wherein the disease is a neurodegenerative disease selected from the group consisting of Alzheimer's disease, dementia, Parkinson's disease, Lou Gehrig disease, motor neuron disease, Huntington's disease and multiple sclerosis.

44. The method according to claim 43 wherein the disease is Parkinson's Disease.

45. A method of treating radiation-induced apoptosis in a mammal said method comprising administering to the mammal an amount of a pharmaceutical formulation according to claim 37 effective to inhibit, delay or prevent radiation-induced apoptosis.

46. A method of treating an adverse effect of a mitochondrial benzodiazepine receptor ligand said method comprising administering to the mammal an amount of a pharmaceutical formulation according to claim 37 effective to inhibit, delay or prevent binding of the ligand to the receptor.

47. The method according to claim 46 wherein the ligand is an agonist of the receptor.

48. The method according to claim 46 wherein the ligand is an antagonist of the receptor.
